(19) United States
(12) Patent Application Publication
(10) Pub. No.: US 2019/0004063 A1
(43) Pub. Date: Jan. 3, 2019

(54) COMPOSITIONS AND METHODS FOR DETECTING SLA REACTIVITY

(71) Applicant: INDIANA UNIVERSITY RESEARCH & TECHNOLOGY CORPORATION, Indianapolis, IN (US)

(72) Inventor: A. Joseph Tector, Vestavia Hills, AL (US)

(21) Appl. No.: 16/023,714

(22) Filed: Jun. 29, 2018

Related U.S. Application Data
(60) Provisional application No. 62/527,245, filed on Jun. 30, 2017.

(51) Int. Cl. G01N 33/68; C12N 5/078; C07K 14/74; C12N 5/0783

(52) U.S. Cl. CPC G01N 33/6854; C12N 5/0634; G01N 2800/245; C12N 5/0636; G01N 2800/50; C07K 14/70539

(57) ABSTRACT

In human-human transplants, the organ recipient's serum is tested against a broad array of HLA (human leukocyte antigens) alleles. The current clinical assay almost performs a virtual crossmatch which allows elimination of incompatible donor organs in advance, but the current assay presents fragments of HLA polypeptides that are not normally visible to antibodies. As a result, the assay yields false positives. Further the current clinical assay is optimized for human to human transplant, not swine to human transplant. Some HLA antibodies also bind swine leukocyte antigens (SLA). Rather than using beads to present antigens, the compositions and methods provide cellular presentation of potential antigens. The application provides a modified human C1R cell line with significantly reduced antigenicity to human sera. Multiple cell lines, each expressing a different SLA, were created. The modified C1R line may express HLAs or other potential antigens of interest. The application also provides modified HEK293T cells for antigen display.

### CROSS-REFERENCE TO RELATED APPLICATIONS

This application claims the benefit of U.S. Provisional Application Ser. No. 62/527,245, filed Jun. 30, 2017 which is incorporated by reference herein as if set forth in its entirety.

### INCORPORATION OF SEQUENCE LISTING

The sequence listing in text format submitted herewith is incorporated by reference in its entirety for all purposes.

### STATEMENT REGARDING FEDERALLY SPONSORED RESEARCH OR DEVELOPMENT

Not applicable.

### FIELD OF THE INVENTION

The present invention relates generally to the field of detecting reactivity to antigens and provides compositions for use in detecting reactivity to presented antigens, particularly swine leukocyte antigens (SLA)s, and methods of detecting reactivity to antigens. In addition the invention provides for methods of predicting whether a transplant recipient has an increased risk for rejection of a transplanted organ or increased risk for developing graft vs. host disease.

### BACKGROUND OF THE INVENTION

Transplants from one animal to another animal of the same species, such as human to human, are a routine treatment option for many serious conditions including kidney, heart, lung, liver and other organ disease and skin damage such as severe burn disease. It is well known that there are not enough suitable organs available for transplant to meet current or expected clinical demands for organ transplants. Approximately 100,000 patients are on the kidney transplant list, and they remain on the waiting list an average of nearly five years before receiving a transplant or dying. In patients with kidney failure, dialysis increases the length of time the patient can wait for a transplant. There is no system comparable to dialysis available for patients with liver disease or liver failure. More than 18,000 patients are on the UNOS liver transplant national waiting list, yet less than 7,000 transplants are performed annually in the United States.

Transplant rejection occurs when the immune system of the transplant recipient attacks the transplanted organ or tissue. The recipient's immune system recognizes the transplanted material as foreign tissue and attempts to destroy it. Rejection occurs through a variety of processes including, but not limited to, antibody mediated rejection. Rejection also occurs when the transplanted organ comprises the donor's lymphocytes or progenitor stem cells which may generate an immune response to the recipient's tissues such as graft vs host disease. Human Leukocyte Antigens (HLAs) are one type of molecules to which a recipient's immune system may respond and cause transplant rejection.

HLA's can bind and display antigens on the surface of human cells to effector T cells. The two major classes of HLA, class I and class II HLAs, present both foreign and native antigens. In response to antigens presented by class I HLA bearing cells, CD8+ effector T cells can destroy the cells. In response to antigens presented by class II HLA bearing cells, CD4+ effector T (helper T cells) cells can mount humoral immune responses. HLAs may be involved in certain cancers and autoimmune disorders as well as transplant rejection.

Antibodies to HLAs are usually produced by alloimmunization resulting from transfusions, pregnancies, transplants or other exposures. Antibodies to HLAs have also been found in non-alloimmunized individuals. See Morales-Buenrostro et al (2008), Transplantation 86:1111-15. Antibodies to HLAs found in transplant recipients have been shown to be a cause of acute and chronic graft rejection. Thus determining whether a recipient carries antibodies to HLAs of a donor can be important in determining the risk of graft rejection in a recipient.

Cell-based assays have been used for cross-matching HLA antigens. These assays are used to determine if a recipient has antibodies in their serum that are cytotoxic to the lymphocytes of a prospective donor. The recipient serum can be frozen prior to the assay. Exemplary standard procedures for cross-matching include complement-dependent cytotoxicity test (CDC), CDC with antiglobulin augmentation (AHG) and flow cytometry cross-match (FC) (Noreen, The American Society for Histocompatibility and Immunogenetics Laboratory Manual, 3^{rd} Ed. I.C.1.1-I.C.1.13.).

The number of known HLA antigens is continuously increasing, and solid-phase HLA antibody detection assays, such as bead-based assays have been developed. The solid-phase assays allow for screening of many HLA antigens in one assay. Therefore many laboratories are using solid-phase HLA antibody detection assays and virtual cross-matching assays in addition to cell-based assays. “Virtual cross-matching” is a procedure that predicts the result of a cell-based cross-match assay. Virtual cross-matching is carried out by comparing the donor antigen profile (donor tissue typing) and the recipient HLA antibody profile as determined by solid-phase assays. For example some laboratories first screen the recipient sera using a solid-phase assay to determine potential donors and subsequently use a cell-based assay for the cross match analysis of the donor cells and recipient sera. Sera that is positive for antibodies specific for the donor HLA antigens in both the solid-phase assay and the cell-based assay is not considered an appropriate match for transplant. However, sera that is positive for antibodies specific for the donor HLA antibodies in the solid-phase assay but negative in the cell-based assay may be showing a false-positive for the HLA antibodies. Commercially available solid-phase assays present many antigens on the beads, including portions of the HLA that are normally hidden inside the donor cell or cell membrane and which are thus not recognized by the recipient's immune system.

Xenotransplantation, the transplant of organs, tissues or cells from one animal into another animal of a different species, such as the transplantation of a pig organ into a human recipient has the potential to reduce the shortage of organs available for transplant, potentially helping thousands of people worldwide. However, xenotransplantation using standard, unmodified pig tissue into a human or other primate is accompanied by rejection of the transplanted tissue. The rejection may be a cellular rejection (lymphocyte mediated) or humoral (antibody mediated) rejection including but not limited to hyperacute rejection, an acute rejection, a chronic rejection, may involve survival limiting thrombocytopenia coagulopathy and an acute humoral xenograft reaction (AHXR). While not being limited by mechanism, both humoral and cellular rejection processes may target MHC molecules. The human hyperacute rejection response to pig antibodies present on transplanted tissue is so strong that the transplant tissue is typically damaged by the human immune system within minutes or hours of transplant into the human. Furthermore, different rejection mechanisms may predominate in an organ-preferred manner. An acute or rapid humoral rejection may begin within minutes of transplant; an acute or rapid cellular rejection may begin within days of the transplant. Both humoral and cellular rejections may also have a slower or chronic rejection phase; the chronic phases may occur for years. See Demetris et al. 1998 “Antibody-mediated Rejection of Human Orthotopic Liver Allografts. A study of liver transplantation across ABO blood group barriers”, Am J. Pathol 132:489-502; Nakamura et al 1993 “Liver allograft rejection in sensitized recipients. Observations in a Clinically Relevant Small Animal Model” Am J. Pathol. 142:1383-91; Furuya et al 1992. “Preformed Lymphocytotoxic Antibodies: the Effects of Class, Titer and Specificity on Liver v Heart Allografts” Hepatology 16:1415-22; Tector et al 2001. “Rejection of Pig Liver Xenografts in Patients with Liver Failure: Implications for Xenotransplantation”, Liver Transpl pp. 82-9; herein incorporated by reference in their entirety. For example, early development of thrombocytopenic coagulopathy is a major factor in non-human primate recipient death following xeno-transplant of a pig liver. Yet, if antibody mediated xenograft rejection is prevented, non-human primate (NHP) recipients of pig kidneys do not develop significant thrombocytopenia nor exhibit clinical manifestations of coagulopathy. See for example Ekser et al. 2012 “Genetically Engineered Pig to Baboon Liver Xenotransplantation: Histopathology of Xenografts and Native Organs” PLoS ONE pp e29720; Knosalla et al 2009, “Renal and Cardiac Endothelial Heterogeneity Impact Acute Vascular Rejection in Pig to Baboon Xenotransplantation”, Am J Transplant 1006-16; Shimizu et a 2012. “Pathologic Characteristics of Transplanted Kidney Xenografts”, J. Am. Soc. Nephrology 225-35; herein incorporated by reference in their entirety.

Pig cells express multiple proteins which are not found in human cells. These include, but are not limited to, α1,3-galactosyltransferase (αGal), cytidine monophosphate-N-acetylneuraminic acid hydroxylase (CMAH) and β1-4 N-acetylgalactosaminyltransferase. Antibodies to the Neu5GC, α-Gal, and Sda-like antigens are present in human blood prior to implantation of xeno-tissue, and are involved in the intense and immediate antibody-mediated rejection of implanted tissue. Additionally pig cells express multiple swine leukocyte antigens (SLAs). Unlike humans, pigs constitutively express class I and class II SLAs on endothelial cells. SLAs and HLAs share considerable sequence homology (Varela et al 2003 J. Am. Soc. Nephrol 14:2677-2683). Anti-HLA antibodies present in human serum prior to implantation of porcine tissue cross-react with SLA antigens on porcine tissues. The SLA cross-reacting antibodies contribute to the intense and immediate rejection of the implanted porcine tissue. SLA antigens may also be involved with the recipient's T-cell mediated immune response. Porcine SLAs may include, but are not limited to, antigens encoded by the SLA-1, SLA-2, SLA-3, SLA-4, SLA-5, SLA-6, SLA-8, SLA-9, SLA-11 and SLA-12 loci. Porcine Class II SLAs include antigens encoded by the SLA-DQ and SLA-DR loci.

As clinicians increasingly turn to xeno-transplant for organ and tissue donations, there is a desire for compositions and methods of identifying potential recipients with existing antibodies to antigens present on donor tissue. Compositions and methods can be used to prevent prospective recipients from being excluded due to false positive signals, for example, when assays screening recipients for antibodies to donor HLAs detect instead antibodies to normally hidden sections of HLA.

### SUMMARY OF THE INVENTION

The present application provides an isolated modified C1R cell comprising disruptions in multiple genes. In various aspects, the modified C1R cell may encompass a disrupted CIITA gene, a disrupted Fc receptor gene and at least one disrupted IgG gene. The IgG gene may be selected from the group of IgG comprising IgG-1, IgG-2, IgG-3 and IgG-4. In various aspects, expression of MHC II, Fc receptor and IgG are reduced. In additional aspects, a modified C1R cell exhibits reduced antigenicity to human serum. The application also provides a cell line comprising a modified C1R cell comprising disruptions in multiple genes.

The present application provides an isolated modified C1R cell comprising a disrupted CIITA gene, a disrupted Fc receptor gene and at least one disrupted IgG gene wherein expression of MHCII, Fc receptor and IgG are reduced wherein the modified cell further comprises an expression vector encoding an antigen of interest, wherein expression of the antigen of interest is increased as compared to expression of the expression of the antigen of interest by an isolated modified C1R cell absent the described expression vector. In various aspects, the antigen of interest may be a selected from the group consisting of porcine, bovine, human, ovine and murine antigens of interest. More particularly the antigen of interest may be selected from the group comprising Class I SLAs, Class II SLAs and HLAs. Yet more particularly the Class I SLA may be selected from the group comprising SLA-1, SLA-2, SLA-3, SLA-5, SLA-6, SLA-7, SLA-8, SLA-12 and the Class II SLA may be selected from the group comprising SLA-DQ and SLA-DR. Still more particularly, the antigen of interest may be selected from the group comprising SLA-1*0702, SLA-1*1201, SLA-1*1301, SLA-2-0202 and SLA-2*1001.

Aspects of the application also provide a panel comprising a first isolated modified C1R cell comprising disrupted CIITA gene, a disrupted Fc receptor gene and at least one disrupted IgG gene wherein expression of MHCII, Fc receptor and IgG are reduced, wherein said first cell further comprises an first expression vector encoding a first antigen of interest, wherein expression of said first antigen of interest is increased as compared to expression of said antigen of interest by an isolated modified C1R cell absent said first expression vector and a second isolated modified C1R cell comprising disrupted CIITA gene, a disrupted Fc receptor gene and at least one disrupted IgG gene wherein expression of MHCII, Fc receptor and IgG are reduced, wherein said second cell further comprises a second expression vector encoding a second antigen of interest, wherein expression of said second antigen of interest is increased as compared to expression of said antigen of interest by an isolated modified C1R absent said second expression vector. The panel may further comprise a third, fourth, fifth, sixth, seventh, eighth, ninth, tenth, twentieth, hundredth or thousandth isolated modified C1R cell comprising disrupted CIITA gene, a disrupted Fc receptor gene and at least one disrupted IgG gene wherein expression of MHCII, Fc receptor and IgG are reduced, further comprising a third, fourth, fifth, sixth, seventh, eighth, ninth, tenth, twentieth, hundredth or thousandth expression vector encoding a third, fourth, fifth, sixth, seventh, eighth, ninth, tenth, twentieth, hundredth or thousandth antigen of interest. The panel may comprise a plurality of isolated modified C1R cell comprising disrupted CIITA gene, a disrupted Fc receptor gene and at least one disrupted IgG gene wherein expression of MHCII, Fc receptor and IgG are reduced, wherein each isolated cell further comprises an expression vector encoding a different antigen of interest. By different antigens of interest is intended an antigen of interest not already expressed by an expression vector in an isolated cell of the composition.

The present application provides methods for detecting antibodies that bind an antigen of interest comprising the steps of contacting a sample with a composition comprising an isolated, modified C1R cell comprising a disrupted CIITA gene, a disrupted Fc receptor gene and at least one disrupted IgG gene wherein expression of MHCII, Fc receptor and IgG are reduced, wherein the isolated cell further comprises an expression vector encoding an antigen of interest wherein expression of the antigen of interest is increased as compared to expression of the antigen of interest by a comparable cell lacking the expression vector, and detecting binding of an antibody to said cell, wherein binding of an antibody to the cell is indicative of a sample comprising antibodies specific for the antigen of interest. In various aspects, the sample may be a serum sample. In aspects of the application, the sample may be obtained from a human subject that is a transplant candidate or a transplant or transfusion recipient. In embodiments of the application, detecting binding of an antibody may be performed using flow cytometry. In embodiments of the application, detecting binding of an antibody may be performed using a secondary antibody.

The present application provides an isolated modified C1R cell comprising disruptions in multiple genes. In various aspects, the modified C1R cell may encompass a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum. The disrupted Class II HLA gene may be selected from the group comprising HLA-DRa, HLA-DRβ, HLA-DQβ and HLA-DPβ. The IgG gene may be selected from the group of IgG comprising IgG-1, IgG-2, IgG-3 and IgG-4. The application also provides a cell line comprising a modified C1R cell comprising a disrupted Class I HLA heavy chain gene, a disrupted B cell Fc receptor gene, at least one disrupted Class II HLA α or β chain gene, and at least one disrupted IgG gene.

The present application provides an isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum, and wherein the modified C1R cell further comprises an expression vector encoding an antigen of interest, wherein expression of the antigen of interest is increased as compared to expression of the expression of the antigen of interest by an isolated modified C1R cell absent the described expression vector. In various aspects, the antigen of interest may be a selected from the group consisting of porcine, bovine, human, ovine and murine antigens of interest. More particularly the antigen of interest may be selected from the group comprising Class I SLAs, Class II SLAs and HLAs. Yet more particularly the Class I SLA may be selected from the group comprising SLA-1, SLA-2, SLA-3, SLA-5, SLA-6, SLA-7, SLA-8, SLA-12 and the Class II SLA may be selected from the group comprising SLA-DQ and SLA-DR. Still more particularly, the antigen of interest may be selected from the group comprising SLA-1*0702, SLA-1*1201, SLA-1*1301, SLA-2-0202 and SLA-2*1001.

Aspects of the application also provide a panel comprising a first and second isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum, wherein the first isolated modified C1R cell comprises an first expression vector encoding a first antigen of interest, and wherein the second isolated modified C1R cell comprises a second expression vector encoding a second antigen of interest, wherein expression of the first antigen of interest in the first cell and expression of the second antigen of interest in the second cell are increased as compared to expression of the antigens of interest by an isolated modified C1R cell absent an expression vector. The panel may further comprise a third, fourth, fifth, sixth, seventh, eighth, ninth, tenth, twentieth, hundredth or thousandth isolated modified C1R cell comprising disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum, further comprising a third, fourth, fifth, sixth, seventh, eighth, ninth, tenth, twentieth, hundredth or thousandth expression vector encoding a third, fourth, fifth, sixth, seventh, eighth, ninth, tenth, twentieth, hundredth or thousandth antigen of interest. The panel may comprise a plurality of isolated modified C1R cells comprising disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum, wherein each isolated cell further comprises an expression vector encoding a different antigen of interest. By different antigens of interest is intended an antigen of interest not already expressed by an expression vector in an isolated cell of the composition.

The present application provides methods for detecting antibodies that bind an antigen of interest comprising the steps of contacting a sample with a composition comprising an isolated, modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and IgG are reduced and wherein the cell exhibits reduced antigenicity to human serum, wherein the isolated modified C1R cell further comprises an expression vector encoding an antigen of interest wherein expression of the antigen of interest is increased as compared to expression of the antigen of interest by an isolated modified C1R cell lacking the expression vector, and detecting binding of an antibody to said cell, wherein binding of an antibody to the cell is indicative of a sample comprising antibodies specific for the antigen of interest. In various aspects, the sample may be a serum sample. In aspects of the application, the sample may be obtained from a human subject that is a transplant candidate or a transplant or transfusion recipient. In embodiments of the application, detecting binding of an antibody may be performed using flow cytometry. In embodiments of the application, detecting binding of an antibody may be performed using a secondary antibody.

The present application provides an isolated HEK293T cell comprising a disruption of the β2-microglobulin gene wherein the cell exhibits reduced class I HLA expression, further comprising a first expression vector encoding a first antigen of interest, wherein expression of the first antigen of interest is increased as compared to expression of the first antigen of interest by an isolated modified HEK293T cell comprising a disruption of the β2-microglobulin gene wherein the cell exhibits reduced class I HLA expression. In aspects of the application, the first expression vector further encodes a second antigen of interest wherein expression of the second antigen of interest is increased as compared to expression of the second antigen of interest by an isolated modified HEK293T cell with reduced class I HLA expression absent the expression vector. The isolated modified HEK293T cell may further comprise a second expression vector encoding CD74 wherein expression of CD74 is increased as compared to expression of CD74 by an isolated HEK293T cell comprising a disruption of the β2-microglobulin gene with reduced class I HLA expression. In aspects of the application, the antigen of interest is selected from the group comprising Class I SLAs, Class II SLAs and HLAs. In various aspects the Class I SLA is selected from the group comprising SLA-1, SLA-2, SLA-3, SLA-5, SLA-6, SLA-7, SLA-8, and SLA-12. In various aspects the Class II SLA is selected from the group comprising SLA-DQ and SLA-DR. In various aspects, the first antigen of interest is a class II SLA α-chain and the second antigen of interest is a class II SLA β-chain. In yet another aspect, the first and second antigens of interest are a pair of Class II SLA α- and β-chain antigens selected from the pairs of class II SLA α- and β-chain antigens comprising DRa*020102 and DRβ1*0404, DRα*020102 and DRβ1*1001, DRα*w04 and DRβ1*0403, DRα*w04 and DRβ1*1001, DQα1*0204 and DQβ1*0303, DQα1*0204 and DQβ1*0601, DQα1*0101 and DQβ1*0303, DQα1*0101 and DQβ1*0601, DRα*020102 and DQβ1*0303, DRα*w04 and DQβ1*0303, DRA1 and DRB1, DRA1 and DRB2, DRA2 and DRB1, DRA2 and DRB2, DQA1 and DQB1, DQA1 and DQB2, DQA2 and DQB1, DQA2 and DQB2, DRA1 and DQB1, DRA1 and DQB2, DRA2 and DQB1, DRA2 and DQB2, DQA1 and DRB1, DQA1 and DRB2, DQA2 and DRB1, and DQA2 and DRB2.

The application also provides a panel comprising a first isolated modified HEK293T cell comprising a disruption of the β2-microglobulin gene wherein the cell exhibits reduced Class I HLA expression, wherein the first isolated cell further comprises a first expression vector encoding a first antigen of interest, wherein expression of the first antigen of interest is increased as compared to expression of that antigen of interest by an isolated modified HEK293T cell comprising a disruption of the β2-microglobulin gene wherein the cell exhibits reduced Class I HLA expression absent the expression vector and a second isolated modified HEK293T cell comprising a disruption of the β2-microglobulin wherein the cell exhibits reduced Class I HLA expression, wherein the second isolated cell further comprises a second antigen of interest and expression of the second antigen of interest is increased as compared to expression of the antigen of interest by an isolated modified HEK293T cell with reduced Class I HLA expression absent the expression vector. In various aspects, the panel may further comprise a third isolated modified HEK293T cell comprising a disruption of the β2-microglobulin gene wherein the cell exhibits reduced Class I HLA expression, wherein the third isolated cell further comprises a third expression vector encoding a third antigen of interest, wherein the third antigen of interest is increased as compared to expression of the antigen of interest by an isolated modified HEK293T cell with reduced class I HLA expression absent the expression vector. The panel may further comprise a plurality of isolated modified HEK293T cells comprising a disruption of the β2-microglobulin gene wherein the cell exhibits reduced Class I HLA expression, wherein the isolated cells further comprise an expression vector encoding a plurality of different antigens of interest. In aspects, the isolated modified HEK293T cells comprising a disruption of the β2-microglobulin gene with reduced class I HLA expression may further comprise a second expression vector encoding CD74, wherein expression of CD74 is increased as compared to expression of CD74 by an isolated HEK293T cell with reduced class I HLA expression.

An embodiment provides a panel comprising a first isolated HEK293T cell with reduced class I HLA expression wherein the first isolated cell further comprises a first expression vector encoding a first and second antigen of interest, wherein expression of the first and second antigens of interest is increased as compared to expression of the antigens of interest by an isolated modified HEK293T cell comprising a disruption of the β2-microglobulin gene wherein said cell exhibits reduced Class I HLA expression absent the expression vector, and a second isolated modified HEK293T cell comprising a disruption of the β2-microglobulin gene wherein the cell exhibits reduced Class I HLA expression, wherein the second isolated cell further comprises an expression vector encoding at least a third antigen of interest, wherein expression of the third antigen of interest is increased as compared to expression of the third antigen of interest by an isolated modified HEK293T cell comprising a disruption of the β2-microglobulin gene wherein the cell exhibits reduced Class I HLA expression absent the expression vector. In aspects of the panel, the expression vector of the second isolated cell further encodes an additional antigen of interest and expression of the additional antigen of interest is increased as compared to expression of the additional antigen of interest by an isolated HEK293T cell comprising a disrupted β2-microglobulin gene wherein the cell exhibits reduced Class I HLA expression absent the expression vector. In various aspects, the panel further comprises a third isolated HEK293T cell with reduced class I HLA expression, wherein the third isolated cell further comprises an expression vector encoding at least one additional antigen of interest, wherein the expression of the additional antigen of interest is increased as compared to expression of the antigen of interest by an isolated HEK293T cell disrupted β2-microglobulin gene wherein said cell exhibits reduced Class I HLA absent the expression vector. In various embodiments the panel may further comprise a plurality of isolated HEK293T cells comprising a disruption of the β2-microglobulin gene wherein said cell exhibits reduced Class I HLA expression wherein the each of the isolated cells further comprises an expression vector encoding a different antigen of interest. In various embodiments of the panel, the antigens of interest may be a pair of Class II SLA α- and β-chain antigens from the pairs of Class II SLA α- and β-chain antigens comprising DRα*020102 and DRβ1*0403, DRα*020102 and DRβ1*1001, DRα*w04 and DRβ1*0403, DRα*w04 and DRβ1*1001, DQα1*0204 and DQβ1*0303, DQα1*0204 and DQβ1*0601, DQα1*0101 and DQβ1*0303, DQα1*0101 and DQβ1*0601, DRα*020102 and DQβ1*0303, DRα*w04 and DQβ1*0303, DRA1 and DRB1, DRA1 and DRB2, DRA2 and DRB1, DRA2 and DRB2, DQA1 and DQB1, DQA1 and DQB2, DQA2 and DQB1, DQA2 and DQB2, DRA1 and DQB1, DRA1 and DQB2, DRA2 and DQB1, DRA2 and DQB2, DQA1 and DRB1, DQA1 and DRB2, DQA2 and DRB1, and DQA2 and DRB2.

The application provides a panel comprising at least one isolated modified C1R cell comprising a disrupted Class I HLA heavy chain gene, a disrupted B Cell Fc receptor gene, at least one disrupted Class II HLA α or β chain gene, and at least one disrupted IgG gene, wherein expression of Class I HLA, MHCII, B cell Fc receptor and an IgG are reduced and wherein the cell exhibits reduced antigenicity to human serum, wherein the isolated modified C1R cell further comprises a first expression vector encoding a first antigen of interest, wherein expression of the first antigen of interest is increased as compared to expression of the first antigen of interest by an isolated modified C1R cell absent the expression vector, and at least one isolated modified HEK293 T cell comprising a disruption of the β2-microglobulin gene wherein the cell exhibits reduced Class I HLA expression wherein said isolated modified HEK293T cell comprising a disruption of the β2-microglobulin gene wherein the cell exhibits reduced Class I HLA expression further comprises a second expression vector encoding a second antigen of interest and wherein expression of said second antigen of interest is increased as compared to expression of said second antigen of interest by an isolated HEK293T cell comprising a disruption of the β2-microglobulin gene wherein the cell exhibits reduced Class I HLA absent said second expression vector.

An embodiment provides a method for detecting antibodies that bind an antigen of interest comprising the steps of: contacting a sample with a composition comprising an isolated HEK293T cell comprising a disruption of the 3-2 microglobulin gene wherein the cell exhibits reduced Class I HLA expression, further comprising an expression vector encoding an antigen of interest wherein expression of said antigen of interest is increased as compared to expression of said antigen of interest by an isolated modified HEK293T cell comprising a disruption of the β2-microglobulin gene wherein cell exhibits reduced Class I HLA expression absent said expression vector, and detecting binding of an antibody to the cell, wherein binding of an antibody to the cell is indicative of a sample comprising antibodies specific for the antigen of interest.

Embodiments of the current application provide a transgenic pig comprising a disrupted α(1,3)-galactosyltransferase, CMAH, β4-galactosyltransferase (β4GalNT2) and SLA gene in the nuclear genome of at least one cell of the pig, wherein expression of α1,3-galactosyltransferase, CMAH, β4GalNT2 and SLA are decreased as compared to a wild-type pig. In an embodiment, the disrupted SLA gene is a disrupted SLA-DQ gene. In an aspect the application provides a transgenic pig comprising a disrupted α(1,3)-galactosyltransferase, CMAH and β4GalNT2 are decreased as compared to a wild-type pig and further comprising an alteration in the SLA-DQ gene sequence, wherein expression of the SLA-DQ gene product is altered as compared to a wild-type pig. In various aspects, the alteration of the SLA-DQ gene product is selected from the group of alterations comprising truncations, insertions, substitutions, substitutions at position 55 and a proline substitution at position 55.

### BRIEF DESCRIPTION OF THE DRAWINGS

FIGS. 1A-1D provide a summary of data obtained from human transplant patient sera incubated with various porcine cells. FIG. 1A shows data obtained from peripheral blood monocytes (PBMC) from triple knockout pigs (αGal, CMAH, β4GALNT2) incubated with human transplant serum (Y-axis). Triple knockout PBMC's were also incubated with human transplant serum that had previously been incubated with wild type porcine cells to eliminate background xenoantigen reactivity (absorbed sera, X-axis). Solid circles indicate sera with elevated binding to the triple knockout PBMC. FIG. 1B shows data obtained from the absorbed sera incubated with PBMC from SLA+ (x-axis) or SLA− porcine PBMC (y-axis). Absorption experiments were repeated with sensitized sera selected to have cPRA>80. FIG. 1C depicts the mean fluorescence index (MFI) of IgG binding of depleted sera incubated with either SLA+ PBMC (light bars) or SLA− PBMC (solid bars). FIG. 1D depicts the MFI of IgM binding of depleted sera incubated with either SLA+ PBMC (light bars) or SLA− PBMC (solid bars).

FIG. 2 presents data obtained from assays involving expression of the indicated SLA allele in either human C1R cells or porcine renal endothelial cells. Transformed cells were incubated with monoclonal anti-SLA or anti-β2M antibodies (shaded histograms). Irrelevant isotype antibodies were used as negative controls (open histograms). The three indicated SLA-1 alleles and the the two indicated SLA-2 alleles were expressed in the C1R cell line and the porcine renal endothelial cells. The SLA-2*0202 allele was expressed and bound by the anti-β2M antibody but not the anti-SLA antibody.

FIGS. 3A-3C present data obtained from pig fibroblasts expressing a human CIITA transgene. FIG. 3A provides histograms of cells cloned after transfection with the CIITA transgene stained with monoclonal antibodies specific for either SLA-DR or SLA-DQ. Grey histograms represent cells with cell surface class II SLA. Open histograms represent a cloned cell that contains the expression vector based on its resistance to the selection marker G418 but which fails to express class II SLA. FIG. 3B provides histograms summarizing the fluorescence intensities of the negative cells when stained with monoclonal antibodies specific for SLA-DR or -DQ (grey histograms) and isotype control antibodies (unfilled histograms). Note the identical fluorescent intensities when cells were stained with monoclonal antibodies specific for SLA-DR or -DQ and isotype control antibodies indicating these negative cells expressed little or no class II SLA protein. FIG. 3C provides an image of gel electrophoresis results of RT-PCR analysis of the class II SLA positive cells with primers specific for SLA-DRa (lane A), SLA-DRβ (lane B), SLA-DQα (lane C) and SLA-DQβ (lane D) genes.

FIGS. 4A-4D summarize flow cytometry results evaluating human antibody binding to either SLA-DR or SLA-DQ. HEK293 T cells were engineered or modified to lack expression of β₂-microglobulin to eliminate cell surface class I HLA proteins. These cells were transfected with individual pairs of DR and DQ alpha (α) and beta (β) chains from the 2 SLA haplotypes present in the fibroblasts used to obtain the data summarized in FIG. 5. All 4 combinations of 2 DRa and 2DRβ transgenes were studied. Four pairs of DQα and DQβ transgenes were also studied. FIG. 4A provides phenotypic analysis of parent HEK cells. White histograms represent background staining of cells with an isotype control antibody. Grey histograms represent staining with antibodies for human β₂ microglobulin, class I HLA and class II HLA (-DR and -DQ). The background traces and the grey histograms overlap significantly. The modified HEK293T cells comprising a disruption of the β2-microglobulin gene exhibit reduced Class I HLA expression. FIG. 4B depicts flow cytometry results obtained from cells stained with antibodies specific for SLA-DR or -DQ to assess cell surface expression of the indicated α/β pairs (grey histograms). White histograms represent negative controls staining of the parent HEK293 cell devoid of β₂-microglobulin and class II SLA. FIG. 4C summarizes IgG binding from sera containing class II HLA reactive immunoglobulin to the modified HEK293 cells expressing SLA-DR or -DQ transgenes. Each cell line described in panel B was stained with 16 different human sera, and the presence of cell associated human IgG was detected with fluorescent goat antihuman IgG. Each column represents an individual serum sample. Each row represents a different cell line. Three sera did not cross-react with either SLA-DR or -DQ (none). Other samples contained antibodies that bound to SLA-DR, SLA-DR and SLA-DQ or to SLA-DQ only. Thirteen sera contained IgG specific for varying numbers of class II HLA proteins. MFI of IgG binding to the different SLA class II expressing cells, SLA (+), and to SLA-deficient cells, SLA(−), were calculated. The SLA(+):SLA(−) ratio is shown in the heat map. One serum only bound cells expressing SLA-DQ and generated MFI 130-fold over HEK293 cells that lacked any class II SLA proteins. The DQ β1 allele appears to be the primary target of antibodies in this particular serum because its pairing with either DQα allele had minimal impact on the level of antigenicity. FIG. 4D provides a correlation between the allele names and their respective notations used in panels A and B.

FIG. 5 depicts a chart summarizing results obtained from evaluating human anti-pig IgG antibody binding from 104 individuals to SLA class II negative and SLA class II positive cells. The mean and the standard deviation of the median fluorescence from human antibody binding are shown. Nineteen sera samples lacked antibodies toward class II HLA and 85 samples contained antibodies specific for class II HLA proteins. The remaining 85 sera showed elevated binding to cells expressing SLA-DR and -DQ when compared to cells which did not express SLA. Sera containing class II HLA reactive antibody also exhibited elevated binding to SLA-DR and -DQ-positive cells relative to human sera lacking class II HLA specific antibodies.

FIGS. 6A-6B summarize results obtained from modified HEK293T cells expressing 10 different class II SLA α and β heterodimers in the presence (solid bars) or absence (empty bars) of pig CD74. Results in FIG. 6A summarize binding to an mAB specific for class II SLA. Results in FIG. 6B summarize binding of CerCLIP.1, an mAB specific for invariant chain peptides associated with class II SLA. Note the significant increase in Ab binding to cells expressing CD74. (p=0.002 for both assays, Wilcoxon matched-pairs sign rank test. Specific alleles making up each α-β combination are in the following tables.

TABLE I
Class II SLA alleles

| SLA Allele | Accession No. |
|---|---|
| DRα*020102 | AIH07184.1 |
| DRα*w04 | AIH07183.1 |
| DRβ1*0403 | AIH07189.1 |
| DRβ1*1001 | AIH07190.1 |
| DQα1*0204 | AIH07186.1 |
| DQα1*0101 | AIH07185.1 |
| DQβ1*0303 | AIH07187.1 |
| DQβ1*0601 | AIH07188.1 |

These alleles represent two haplotypes from a single pig (8). The accession numbers were obtained from GenBank.

TABLE II
Allele combination nomenclature

| SLA Allele Combination | Pairing |
|---|---|
| DRα*020102 + DRβ1*0403 | 1 |
| DRα*020102 + DRβ1*1001 | 2 |
| DRα*w04 + DRβ1*0403 | 3 |
| DRα*w04 + DRβ1*1001 | 4 |
| DQα1*0204 + DQβ1*0303 | 5 |
| DQα1*0204 + DQβ1*0601 | 6 |
| DQα1*0101 + DQβ1*0303 | 7 |
| DQα1*0101 + DQβ1*0601 | 8 |
| DRα*020102 + DQβ1*0303 | 9 |
| DRα*w04 + DQβ1*0303 | 10 |

The various pairings of the α- and β-chains of DR and DQ are indicated. Pairing numbers will be used to simplify the labeling of figures.

Cell lines were also stained with the mAb CerCLIP.1. The CerCLIP.1 peptide binding groove of class II HLA molecules. In the absence of CD74, the class II positive cell lines exhibited a background MFI of approximately 1000. Co-expression of invariant chain and class II SLA increased CerCLIP.1 binding in all SLA molecules tested except for one hybrid (pair 9). CerCLIP.1 can be used to probe cell surface expression of class II SLA molecules.

FIGS. 7A-7D provide results obtained from analysis of human antibody reactivity toward class II SLA. 38 HLA unsensitized human sera and 26 HLA sensitized sera were evaluated. FIG. 7A shows results of IgM binding with unsensitized sera; FIG. 7B shows results of IgM binding with sensitized sera; FIG. 7C shows results of IgG binding unsensitized sera; FIG. 7D shows results of IgG binding sensitized sera. The dot plots show the log₁₀ of MFI ratios comparing IgG binding to SLA-positive cells versus SLA-deficient parent cells. Values greater than 0 indicate more binding to class II-positive cells. Log₁₀ values less than 0 indicate greater IgG binding to the parent cell line. When antibodies bind equally to both cell types the log₁₀ value=0. Heat maps below the dot plots show binding results of sera having at least a single log₁₀ greater than or equal to 1. Each heat map row represents an individual serum tested against every class II SLA heterodimer. Grey squares represent class II (+) versus class II (−) MFI ratios less than or equal to 1. More intensely shaded squares indicate increased binding to class II-expressing cells. DR indicates SLA-DRα/DRβ pairs, DQ represents SLA-DQα/DQβ pairs and H represents SLA-DRα/DQβ pairs.

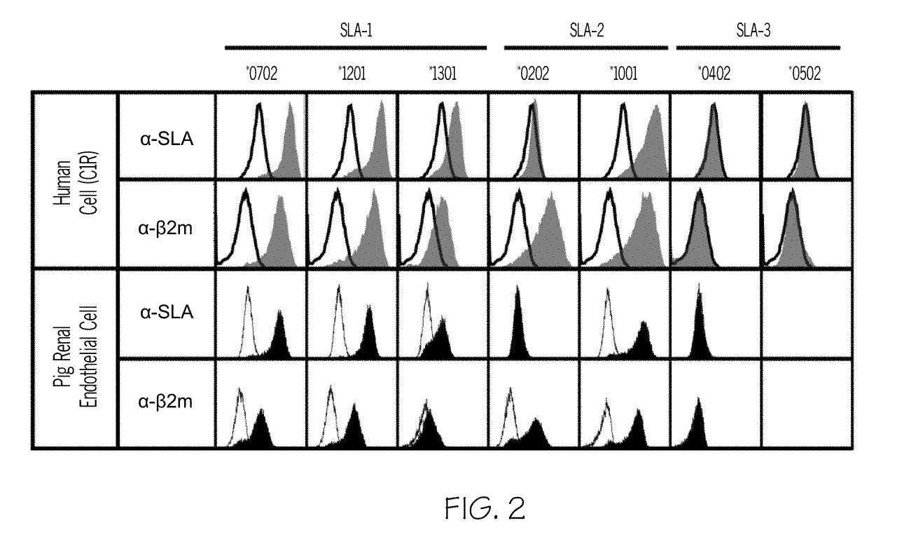

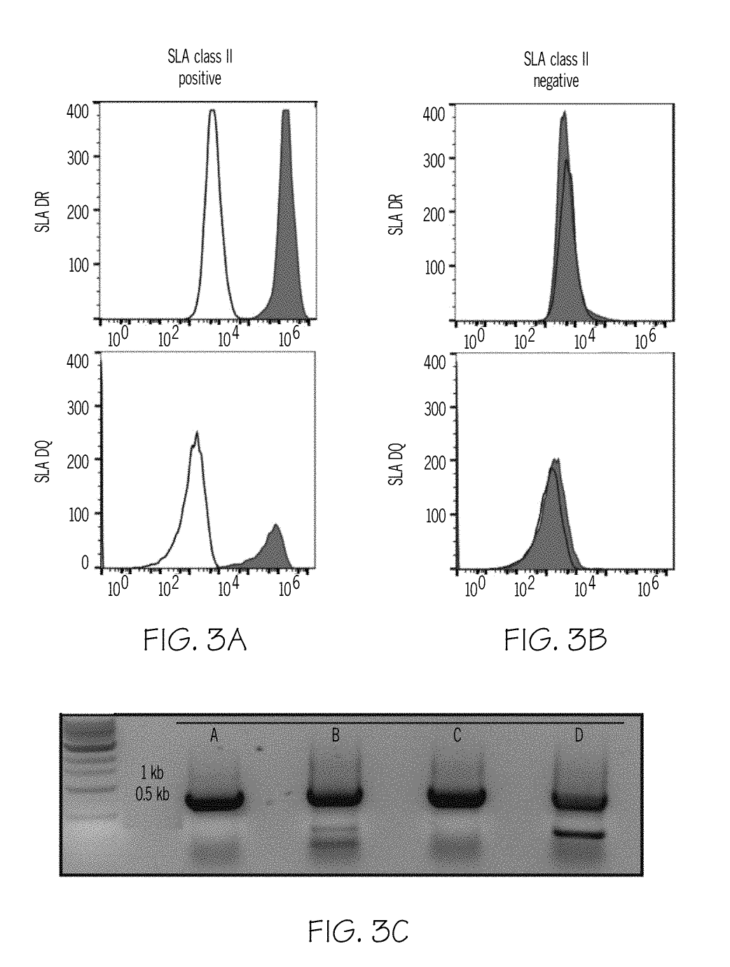

As the dot blots in FIG. 7D indicate in the presence of invariant chain, IgG from sera containing class II HLA Abs frequently cross-reacted with class II SLA. The dotblots in FIG. 7C indicated sera lacking anti-HLA Abs occasionally contained IgG capable of recognized class II SLA. The dot blots in FIGS. 7A and B indicate multiple human serum samples contained IgM capable of binding class II SLA molecules regardless of the presence of class II HLA Abs (FIG. 2A, 2B). Hybrid SLA-DR-α and DQβ molecules did not yield obvious IgM binding but were bound by IgG. Some serum samples contained antibodies (Abs) reactive with most class II SLA proteins, whereas others exhibited a more restricted pattern.

FIGS. 8A-8E provide dot blots summarizing results obtained from evaluations of antibody (Ab)-initiated complement cytotoxicity (percentage killing) by sera with or without class II HLA antibodies. FIG. 8A summarizes data from cells lacking Class II HLA antibodies; FIG. 8B summarizes data from cells with class II HLA antibodies; FIG. 8C summarizes data from cells lacking Class II HLA antibodies, treated with dithiothreitol (DTT); FIG. 8D summarizes data from cells with Class II HLA antibodies, treated with DTT. DR indicates SLA-DRα/DRβ pairs, DQ represents SLA-DQα/DQ pairs and H represents SLA-DRα/DQβ pairs. Parent represents killing against cells lacking both class II SLA proteins and CD74. FIG. 8E summarizes results obtained from experiments with a single serum against cells expressing all 10 class II SLA proteins before and after DTT treatment (±DTT comparison p=0.001, Wilcoxon test).

Residual lytic activity, despite DTT-mediated inactivation of IgM, showed that IgG specific for class II SLA drove cytotoxicity (FIGS. 8C and 8D). The decrease of cell death in the presence of DTT (FIG. 8E) indicates IgM specific for class II SLA also killed cells.

FIG. 9 provides graphs of the MFI of antibody binding from six human sera to beads containing the different HLA-DQ proteins. These antibodies were eluted from cells expressing class II SLA's. Four of the six sera contained cross-reactive antibodies primarily recognizing HLA-DQ4,5, 6. The other sera recognized either HLA-DQ2, 4 or HLA-DQ2,4,6,7,8,9. No cross-reactivity between SLA-DQα1*0101/DQβ1*0601 molecules and HLA-DR or HLA-DP was detected.

FIGS. 10A-10B-FIG. 10A depicts a structural prediction of the SLA-DQα1*01001/DQβ1*0601 and HLA-DQ4,5,6 polypeptides. The modeling was based on sequence alignments of the polypeptides. Modeling suggests the arginine at position 55 is involved with antibody binding. The 55R residue was mutated to a proline. The 55P mutation reduced but did not eliminate SLA reactivity with the human sera. FIG. 10B summarizes the MFI results obtained from analysis of human IgG binding to a cell expressing CD74, SLA-DQα1*0101 and DQβ1*0601. All tested sera contained some class II HLA-binding IgG. The samples were split into two groups based on the presence (n=42) or absence (n=21) of HLA-DQ4,5,6 reactivity. The presence of Abs against HLA-DQ4,5,6 increased binding to SLA-DQα1*0101 and DQβ1*0601 (Mann-Whitney U test p=0.030).

FIGS. 11A-11B provide summaries of experiments investigating impact of an R55P mutation in SLA-DQ3. FIG. 11A provides histograms of binding of the CerCLIP.1 Ab to cells expressing either 55R or 55P. Filled histograms show CerCLIP.1 binding to the CD74/SLA-deficient HEK293T cells. FIG. 11B depicts results from 21 human sera, each represented by one red and one black bar. MFI ratios of the fluorescent anti-human IgG were calculated to compare binding to 55P versus 55R molecules. The log₁₀ values of these ratios were plotted for each serum (black bars). The same analysis compared IgG binding to the 55P cells versus SLA-negative parent cells (red bars). The 55P mutation partially reduced IgG binding to the SLA-DQ protein in 17 samples. Three samples had no detectable antibody staining of 55P mutants. One sample showed greater binding to the 55P mutant than to 55R. The mAB sample indicates the binding of a mAb specific for SLA-DQ. This antibody recognized SLA-DQ 55P variants (red bar) almost identically to 55R variants (black bar, log₁₀ of 55P/55R=−0.09.)

FIGS. 12A-12B provide flow cytometry traces of HEK293 SLA-DR and DQ panel. Cells were stained with antibodies specific for (FIG. 12A) SLA-DR or -DQ or (FIG. 12B)-CerClip.1 to validate cell surface expression of the indicated α-β pairs (empty traces) in the presence or absence of CD74. Shaded histograms represent negative control staining of the parent modified HEK293 cell devoid of β2-microglobulin and class II SLA. Allele pairing is set forth in Table II, elsewhere herein. DR indicates DRα/DRβ pairs, DQ indicates DQα/DQβ pairs and hybrid represents DRα/DQβ pairs. Analysis of these data is further provided in FIGS. 13A-13B.

FIGS. 13A-13B provide a histogram showing the percent expression of SLA class II from library of modified HEK293T cell transfected with 16 different SLA class II α-β plasmid combinations with (red) or without (black) dual transfection of the swine invariant chain. The traditional cis and trans-haplotype pairings are shown in FIG. 13A; the hybrid haplotype pairings are shown in FIG. 13B.

FIGS. 14A-14C provide information relating to the modified C1R cell line comprising a disrupted Class I HLA heavy chain gene, a disrupted B cell Fc receptor gene, at least one disrupted Class II HLA α or β chain gene and at least one disrupted IgG gene for antibody analysis. FIG. 14A provides a cartoon of various epitopes found on an unmodified C1R lymphoblast cell and antibodies that bind thereto. Endogenous IgG, Fc Receptor, Class I HLA, Class II HLA, Serum Ig, Neu5Gc and anti-human Fab′2 are represented by the indicated symbol. FIG. 14B provides a graphic representation of the effect of the genetic modifications on the engineered C1R cell line. The starting, unmodified C1R cell is bound by many human antibodies. After CRISPR/Cas9 modification of the Fc receptor gene, a Class I HLA heavy chain gene, a Class II HLA gene, and an IgG gene, many of the complicating epitopes were no longer present on the modified C1R cell membrane. Growth in Neu5Gc deficient cell media reduces the amount of Neu5Gc on the modified C1R cell, thus further reducing antibody binding. Background binding is further reduced by non-specific IgM binding blocking. FIG. 14C provides histograms showing of antibody binding to the modified C1R cell line and the starting, unmodified C1R cell line. The shaded histogram is an isotype control and the dark black line indicates monoclonal or polyclonal antibody binding by anti-β2microglobulin (α-B2M, HLA Class I), anti-CII (αCII, HLA Class II), anti-IgG (α-IgG, IgG in BCR), anti-Fc receptor (α-Fcy, Fc-receptor), anti-Neu5Gc (α-HD, Neu5gc uptake) and anti-IgM (α-IgM, IgM binding) antibodies.

FIGS. 15A-15D provide schematics and data obtained from experiments involving expression of single HLA/SLA antigens in an isolated modified C1R cell comprising a disrupted Class I HLA heavy chain gene, a disrupted B cell Fc receptor gene, at least one disrupted Class II HLA α or β chain gene and at least one disrupted IgG gene, wherein expression of Class I HLA, MHCII, B cell Fc receptor and an IgG are reduced. The terms “engineered C1R” and “modified C1R” cells refer to genetically altered or modified C1R cells. FIG. 15A provides a cartoon depiction of a modified C1R cell before (first frame), after transfection with an expression vector producing a single HLA molecule (second frame) and after incubation with human antibody binding (third frame). FIG. 15B provides flow cytometry traces of engineered C1R cells transfected with an expression vector comprising the indicated HLA allele after incubation with anti-β2-microglobulin (thick line traces). The shaded areas represents flow cytometry traces of engineered C1R cells transfected with an empty expression vector. FIG. 15C provides heat maps of binding to HLA expressing engineered C1R cells (e-C1R) compared to luminex single antigen bead binding (Luminex) by sera from 5 sensitized patients. White cells indicate minimal antibody binding over background in each assay and dark boxes represent high antibody binding in each assay. FIG. 15D provides flow cytometry traces of engineered C1R cells transfected with an expression vector comprising the indicated SLA allele after incubation with anti-β2-microglobulin (thick line traces). The shaded areas represents flow cytometry traces of engineered C1R cells transfected with an empty expression vector. SLA*3-4 is not expressed in the engineered C1R cells (data not shown).

FIGS. 16A-16E provide data obtained from experiments involving expression of single HLA/SLA antigens in an isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and an IgG are reduced, and wherein said cell exhibits reduced antigenicity to human serum. FIG. 16A depicts a heat map of IgG antibody binding from representative (n=11) sensitized patient sera represented at ratio over empty cell binding (range 1-60). FIG. 16B provides a chart summarizing multiple unique epitopes include 144 Lysine/K and these amino acids are conserved in some or all known pig alleles. FIG. 16C depicts a model of the SLA epitopes. The location of the lysine residue at position 144 is circled. FIG. 16D provides flow cytometry traces obtained after incubation of engineered C1R cells expressing either SLA*1-12 or HLA*A3 with either lysine (K) or glutamine (Q) with α-β2 microglobulin antibodies. FIG. 16E provides graphs summarizing human IgG binding to the native (144K) and mutated (144Q) cells are shown as the ratio to background binding.

### DETAILED DESCRIPTION OF THE INVENTION

The present application provides a genetically modified C1R cell line with reduced expression of MHC type I, MHC type II, Fc receptor and IgG. The genetically modified “naked” C1R human cells show significantly reduced antigenicity. The terms “genetically modified” or “engineered” are used similarly. When incubated with the modified C1R cells, human sera shows significantly reduced antibody binding indicating the modified C1R cells have reduced antigenicity or capacity to act as an antigen. Modified C1R cells are transfected with expression vectors comprising different SLAs. The transfected modified C1R cells express the antigen of interest from the expression vector. When human serum is incubated with the modified C1R cell expressing an antigen of interest, binding indicates the presence of antibodies to the antigen of interest. Testing a candidate transplant recipient serum against the antigen of interest expressed on the modified C1R cell indicates whether the candidate transplant recipient already expresses antibodies to the donor material and prevents a transplant candidate from receiving an unsuitable organ or tissue. Additionally the methods and compositions can be used to evaluate whether a transplant recipient has developed antibodies to the donor organ or tissue.

### I. In General

In the specification and in the claims, the terms “including” and “comprising” are open-ended terms and should be interpreted to mean “including, but not limited to . . . ” These terms encompass the more restrictive terms “consisting essentially of” and “consisting of”.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. Compositions and Methods

The “modified C1R” cell line of the current application was developed from the human B cell derived cell line, C1R. The C1R cell line is known to have Class I MHC expression reduced. The C1R cell is described in Storkus et al (1989) Proc Natl Acad Sci 86:2361-2364 and Zemmour et al (1992) J. Immunol 148:1941-1948, herein incorporated by reference in their entirety. C1R cells are available from the ATCC. The engineered or modified c1R cell line and its preparation is described elsewhere herein. Modified HEK293T cells with reduced Class I HLA expression include any HEK293T cell engineered to exhibit reduced expression of one or more Class I HLA. HEK293T cells with reduced Class I HLA expression may include, but are not limited to HEK293T cells comprising a disrupted β2-microglobulin gene wherein expression of β2-microglobulin is reduced.

The term “disrupted” in reference to a gene is intended to encompass insertion, interruption, or deletion of a nucleotide sequence of interest wherein the disrupted gene either encodes a polypeptide having an altered amino acid sequence that differs from the amino acid sequence of the endogenous sequence, encodes a polypeptide having fewer amino acid residues than the endogenous amino acid sequence or does not encode a polypeptide although the wild-type nucleotide sequence of interest encodes a polypeptide. Methods of disrupting a gene or gene-editing techniques are known in the art and include, but are not limited to, CRISPR-CAS9, TALENS, Zinc-fingers, CRISPR-Cas9 based systems, and CRISPR-CPf1. Any method of disrupting a gene sequence known in the art may be used.

By “pig” is intended any pig known to the art including, but not limited to, a wild pig, domestic pig, mini pigs, a Sus scrofa pig, a Sus scrofa domesticus pig, as well as in-bred pigs. Without limitation the pig can be selected from the group comprising Landrace, Yorkshire, Hampshire, Duroc, Chinese Meishan, Chester White, Berkshire Goettingen, Landrace/York/Chester White, Yucatan, Bama Xiang Zhu, Wuzhishan, Xi Shuang Banna and Pietrain pigs. Porcine organs, tissues or cells are organs, tissues, devitalized animal tissues, or cells from a pig.

Swine produce swine leukocyte antigens (SLA) from multiple MHC genes. The SLA molecules are homologs of human leukocyte antigens (HLA), a major determinant of allotransplant success. Class I HLA and SLA proteins consist of a polymorphic membrane bound MHC protein associated with the invariant β2-microglobulin (CD74) and short peptide fragment. Class II proteins contain MHC-encoded polymorphic membrane-bound α and β-chains and a short peptide (Blum et al 2013, Annu Rev. Immunol 31:443-473, herein incorporated by reference in its entirety.) Multiple Class I and Class II MHC genes, with each locus containing many alleles, contribute to significant variation among HLA and SLA proteins. Nonetheless HLA and SLA proteins contain significant structural and amino acid sequence identity. Swine CD74 encodes the swine invariant chain; “CD74” and “invariant chain” refer to the same polypeptide.

Humans and non-human primate CD8+ and CD4+ T cells can be activated by SLA Class I and II, respectively. SLA's are characterized in a class selected from the group comprising Class I and Class II. SLA genes include, but are not limited to SLA-1, SLA-2, SLA-3, SLA-4, SLA-5, SLA-9, SLA-11, SLA-DQ and SLA-DR. SLA-1, SLA-2 and SLA-3 are SLA Class I (SLA1) genes. SLA-DQ and SLA-DR are SLA Class II genes. Anti-SLA Class 1 (anti-SLA1) antibodies may react with products of the SLA-1, SLA-2 and SLA-3 genes. The SLA-1*0702 allele sequence is available as Genbank Acc. No: EU440330.1. The SLA-1*1201 allele sequence is available as Genbank Acc. No: EU440335.1. The SLA-1*1301 allele sequence is available as Genbank Acc. No: EU440336.1. The SLA-2 1001 allele sequence is available as Genbank Acc. No: EU432084.1. The SLA-2 2002 allele sequence is available as Genbank Acc. No: EU432081.1. The SLA-3*0402 allele sequence is available as Genbank Acc. No: EU432092.1. The SLA-3*0502 allele sequence is available as Genbank Acc. No: EU432094.1. Any SLA allele known in the art may be expressed in the modified C1R or HEK293T cells of the current application. Identified SLA alleles are described on the IPD-MHC Swine Leukocyte Antigen website http://www.ebi.ac.uk/ipd/mhc/group/SLA.

Pig CD8+ T cells constitutively express Class II SLA. Further pigs contain abundant circulating CD4+/CD8+ double positive T cells; thus, isolating a specific population of porcine lymphocytes lacking class II MHC is challenging. Crossmatches of wildtype red blood cell absorbed sera on swine peripheral blood monocytes (PBMCs) remove the unknown antiglycan xenoantibodies but leaves anti-Class I SLA antibodies, making interpretation difficult. Class II SLA is like HLA in that pigs express DR and DQ, but is distinct from HLA in that there is no DP. The human CIITA transcription factor alters HLA Class II expression in human cells; it also regulates several additional genes in addition to Class II MHC (MHCII) molecules. In porcine cells the human CIITA transcription factor transgene allows expression of the SLA class II genes, -DR and -DQ. Anti-DQ antibodies are among the most frequent HLA antibodies found in recipients with a failed renal allograft. An additional Class II SLA expression system which avoids the use of CIITA was developed.

The compositions provided herein may comprise SLA of the same allele or two or more different alleles. As used herein, the term “same SLA allele” refers to two or more SLA molecules or fragments thereof that share similar structure and antigenic properties and are derived from the same SLA gene loci and alleles. As used herein, the term “different SLA alleles” refers to SLA molecules or fragments thereof that possess different structure and antigenic properties and are derived from different SLA gene loci and alleles.

Initial pig-to-human transplantation experiments failed because preformed antibodies (Abs) recognized glycan antigens on the donor tissue. Since then pigs have been engineered to prevent expression of genes such as α1,3-galactosyltransferase (GGTA1), cytidine monophosphate-N-acetylneuraminic acid hydroxylase (CMAH) and β-1,4-N-acetyl-galactosaminyltransferase 2 (β4GalNT2). Eliminating multiple xenoantigens from pigs may reduce Ab-mediated rejection of pig xenotransplant organs. Human antibodies also react to porcine Class II SLA's. Eliminating Class II SLA's or eliminating common epitopes from porcine Class II SLA's may reduce antibody mediated rejection of pig organs and increase the success of xenotransplant. Careful histocompatibility analyses that match pig donors and human recipients may improve xenotransplant success by preventing transplant and subsequent rejection of a pig organ to which the human recipient already has donor-specific antibodies. We provide a panel or library of cells expressing antigens, such as, but not limited to, Class I SLA antigens, Class II SLA antigens and HLA's which can be used to assess a potential recipient's serum for donor specific antigens. Successfully targeting xenotransplantation of donor organs to recipient humans may improve a xenotransplant rejection related symptom, particularly an antibody-mediated rejection related symptom.

“Xenotransplantation” encompasses any procedure that involves the transplantation, implantation or infusion of cells, tissues or organs into a recipient subject from a different species. Xenotransplantation in which the recipient is a human is particularly envisioned. Thus xenotransplantation includes but is not limited to vascularized xenotransplant, partially vascularized xenotransplant, unvascularized xenotransplant, xenodressings, xenobandages, xenotransfusions, and xenostructures.

Transplant rejection occurs when transplanted tissue, organs, cells or material are not accepted by the recipients' body. In transplant rejection, the recipient's immune system attacks the transplanted material. Multiple types of transplant rejection exist and may occur separately or together. Rejection processes included but are not limited to hyperacute rejection (HAR), acute humoral xenograft rejection reaction (AHXR), thrombocytopenia, acute humoral rejection, hyperacute vascular rejection, antibody mediated rejection and graft versus host disease. By “hyperacute rejection” we mean rejection of the transplanted material or tissue occurring or beginning within the first 24 hours post-transplant involving one or more mechanisms of rejection. Rejection encompasses but is not limited to “hyperacute rejection”, “humoral rejection”, “acute humoral rejection”, “cellular rejection” and “antibody mediated rejection”. The acute humoral xenograft reaction (AHXR) is characterized by a spectrum of pathologies including, but not limited to, acute antibody mediated rejection occurring within days of transplant, the development of thrombotic microangiopathy (TMA), microvascular angiopathy, pre-formed non-Gal IgM and IgG binding, complement activation, microvascular thrombosis and consumptive thrombocytopenia within the first few weeks post transplant. Thrombocytopenia is a quantity of platelets below the normal range of 140,000 to 440,000/μl. Thrombocytopenia related symptoms include, but are not limited to, internal hemorrhage, intracranial bleeding, hematuria, hematemesis, bleeding gums, abdominal distension, melena, prolonged menstruation, epistaxis, ecchymosis, petechiae or purpura. Uptake of human platelets by pig livers contributes to the development of thrombocytopenia in xenograft recipients. Thrombocytopenia may occur upon reperfusion of the xenotransplanted organ or after the immediate post-reperfusion period. Additional pathologies include, but are not limited to, transplant glomerulopathy and graft loss.

Transgenic transplant material. Transplant material encompasses organs, tissue and/or cells from an animal for use as xenografts. Transplant material for use as xenografts may be isolated from transgenic animals with decreased expression of αGal, SLA and CMAH or from other transgenic animals. Transgenic transplant material from transgenic pigs can be isolated from a prenatal, neonatal, immature or fully mature animal. The transplant material may be used as temporary or permanent organ replacement for a human subject in need of an organ transplant. Any porcine organ can be used including, but not limited to, the brain, heart, lung, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, small bowel, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes and lymph vessels.

Expression of a gene product is decreased when total expression of the gene product is decreased, a gene product of an altered size is produced or when the gene product exhibits an altered functionality. Thus if a gene expresses a wild-type amount of product but the product has an altered enzymatic activity, altered size, altered cellular localization pattern, altered receptor-ligand binding or other altered activity, expression of that gene product is considered decreased. Expression of antigen of interest is increased when total expression of the antigen of interest is increased, even if the expressed antigen of interest does not perform its native function. Expression may be analyzed by any means known in the art including, but not limited to, RT-PCR, Western blots, Northern blots, microarray analysis, immunoprecipitation, radiological assays, polypeptide purification, spectrophotometric analysis, Coomassie staining of acrylamide gels, ELISAs, 2-D gel electrophoresis, in situ hybridization, chemiluminescence, silver staining, enzymatic assays, ponceau S staining, multiplex RT-PCR, immunohistochemical assays, radioimmunoassay, colorimetric assays, immunoradiometric assays, positron emission tomography, fluorometric assays, fluorescence activated cell sorter staining of permeablized cells, radioimunnosorbent assays, real-time PCR, hybridization assays, sandwich immunoassays, flow cytometry, SAGE, differential amplification or electronic analysis. Expression may be analyzed directly or indirectly. Indirect expression analysis may include but is not limited to, analyzing levels of a product catalyzed by an enzyme to evaluate expression of the enzyme. See for example, Ausubel et al, eds (2013) Current Protocols in Molecular Biology, Wiley-Interscience, New York, N.Y. and Coligan et al (2013) Current Protocols in Protein Science, Wiley-Interscience New York, N.Y.

“As compared to” is intended to encompass comparing something to a similar but separate thing, such as comparing a data point obtained from an experiment with a transgenic pig to a data point obtained from a similar experiment with a wildtype pig. The word “comparing” is intended to encompass examining character, qualities, values, quantities, or ratios in order to discover resemblances or differences between that which is being compared. Comparing may reveal a significant difference in that which is being compared. By “significant difference” is intended a statistically significant difference in results obtained for multiple groups such as the results for material from a transgenic pig and material from a wild-type pig or results for material from a triple transgenic product or pig and material from a double transgenic product or pig. Generally statistical significance is assessed by a statistical significance test such as but not limited to the student's t-test, Chi-square, one-tailed t-test, two-tailed t-test, ANOVA, Dunett's post hoc test, Fisher's test and z-test. A significant difference between two results may be results with a p<0.1, p<0.05, p<0.04, p<0.03, p<0.02, p<0.01 or greater.

The word “isolated” is intended to encompass an entity that is physically separated from another entity or group. An isolated cell is physically separated from another group of cells. Examples of a group of cells include, but are not limited to, a developing cell mass, a cell culture, a cell line, a tissue, an organ and an animal. The word “isolating” is intended to encompass physically separating an entity from another entity or group. Examples include physically separating a cell from other cells, physically separating a cell component from the remainder of the cell and physically separating tissue or organ from an animal. An isolated cell or cell component is separated by 10%, 15%, 20%, 25%, 30%, 35%, 40%, 45%, 50%, 55%, 60%, 65%, 70%, 75%, 80%, 85%, 90%, 95%, 96%, 97%, 98%, 99%, up to 100% of other naturally occurring cells or cell components. Methods for isolating one or more cells from another group of cells are known in the art. See for example Freshney (ED) Culture of Animal Cells: a manual of basic techniques (3^{rd} Ed.) 1994, Wiley-Liss; Spector et al (Eds)(1998) Cells: a Laboratory Manual (vol. 1) Cold Spring Harbor Laboratory Press and Darling et al (1994) Animal Cells: culture and media John Wiley & Sons. Methods of isolating a tissue or an organ from an animal are known in the art and vary depending on the tissue or organ to be isolated and the desired method of transplanting the tissue or organ. Methods of isolating a transfusion product from an animal or sample are known in the art and vary depending on the desired transfusion product. Such methods include but are not limited to centrifugation, dialysis, elution, apheresis and cryoprecipitation.

Antigens of interest may be a whole protein, a truncated protein, or a fragment of a protein or a peptide. Antigens may be naturally occurring, genetically engineered variants of the protein, or may be codon optimized for expression in a particular mammalian subject or host. Generally a B-cell epitope will include at least about 5 amino acids but can be as small as 3-4 amino acids. SLA and HLA antigens may be recombinantly expressed and presented on the naked or modified C1R cell surface. Normally an epitope will include between about 7 and 15 amino acids, such as, 9, 10, 12, or 15 amino acids. The term “antigen” denotes both subunit antigens (i.e. antigens which are separated and discrete from a whole organism with which the antigen is associated in nature). Antibodies such as anti-idiotype antibodies, or fragments thereof and synthetic peptide mimotopes, that is synthetic peptides which can mimic an antigen or antigenic determinant, are also captured under the definition of antigen as used herein.

Furthermore for purposes of the present application, an “antigen” refers to a protein which includes modifications such as deletions, additions, and substitutions, generally conservative in nature, to the naturally occurring sequence, so long as the protein maintains the ability to elicit an immunological response. These modifications may be deliberate as through site-directed mutagenesis, or may be accidental, such as through mutations of hosts which produce the antigens. Antigens may also be codon optimized by methods known in the art to improve their expression or immunogenicity in the host.

Biological samples include, but are not limited to, whole blood, blood derivatives, red blood cell concentrates, plasma, serum, fresh frozen plasma, whole blood derived platelet concentrates, globulin, apheresis platelets, pooled platelets, intravenous gamma-globulin, cryoprecipitate, cerebrospinal fluid, tissues and cells such as epithelial cells, such as those collected from the buccal cavity, stem cells, leukocytes, neutrophils and granulocytes. The biological sample may be obtained from any human donor, but more particularly from a human subject that is an intended recipient of a transplant or transfusion, transplant or transfusion candidate, or transplant or transfusion recipient. A transplant candidate is a human subject in need of transplanted material such as an organ, organs, tissue, tissues or cells prior to the subject receiving the transplant. A transplant or transfusion recipient is a subject who has previously received or is concurrently receiving a transplant or transfusion. Monitoring the transplant or transfusion recipient's reaction to donor specific antigens improves timely clinical interventions to decrease a rejection related symptom.

The methods may be performed with any method of detecting serum cell binding known in the art including, but not limited to, flow cytometry, immunoblotting and secondary antibody techniques. Exemplary secondary antibodies include those which comprise a label selected from the group comprising a radioactive label, fluorescent label, enzymatic label, avidin label, biotin label, magnetic labels and protein tags. Any reagent useful for detection of antibody binding known to those of skill in the art can be used. Secondary antibodies, for example, can be useful for the detection of antibody binding in assays such as Western blots, immunoblots, flow cytometry, ELISAs and radioimmunoassays (RIAs).

Expression vectors are known in the art. Any expression vector suitable for use in the indicated cell line may be used in the instant methods and compositions. Expression vectors may include regulatory regions, antibiotic resistance regions, selection mechanisms, replication origins and other components.

In another embodiment, the application provides non-human tissues that are useful for xenotransplantation. In various embodiments, the non-human tissue is porcine tissue from a quadruple αGal/CMAH/β4GalNT2/SLA transgenic pig. Any porcine tissue can be used including but not limited to, epithelium, connective tissue, blood, bone, cartilage, muscle, nerve, adenoid, adipose, areolar, brown adipose, cancellous muscle, cartilaginous, cavernous, chondroid, chromaffin, dartoic, elastic, epithelial, fatty, fibrohyaline, fibrous, Gamgee, gelatinous, granulation, gut-associated lymphoid, skeletal muscle, Haller's vascular, indifferent, interstitial, investing, islet, lymphatic, lymphoid, mesenchymal, mesonephric, multilocular adipose, thymus tissue, mucous connective, myeloid, nasion soft, nephrogenic, nodal, osteoid, osseus, osteogenic, bone marrow, retiform, periapical, reticular, smooth muscle, hard hemopoietic and subcutaneous tissue, devitalized animal tissues including heart valves, skin, and tendons, and vital porcine skin.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

The following examples are offered by way of illustration and not limitation.

### EXPERIMENTAL

Although the foregoing invention has been described in some detail by way of illustration and example for purposes of clarity of understanding, it will be obvious that certain changes and modifications may be practiced within the scope of the appended claims.

### Example. 1 Human Sera Cross Reacts with SLAs

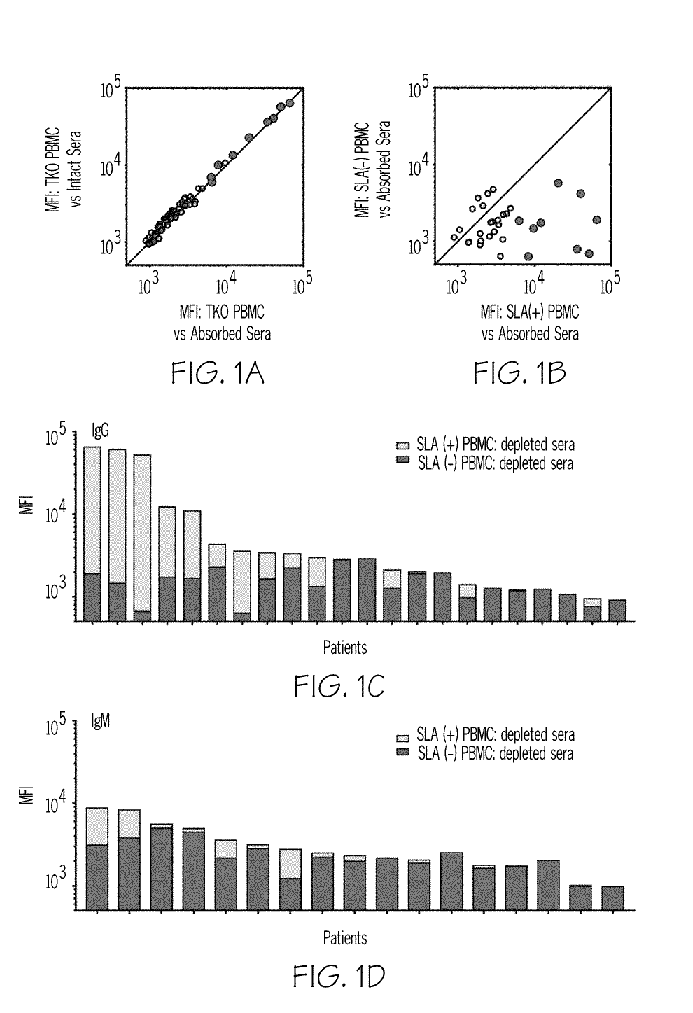

IgG binding to peripheral blood monocytes (PBMC) from triple knockout (TKO) pigs lacking CMAH, αGal and β4GalNT2 before and after absorbing each serum on wild-type (VVT) pig red blood cells (RBC) was analyzed using flow cytometry. The MFI of 73 sera preabsorption and postabsorption varied less than 25%. Data from the sera that exhibited more than 25% variation are not shown. Ten sera retained dramatically increased levels of binding to TKO cells (MFI>5×10³) indicating an abundance of reactivity to antigens not on RBCs. Data from an exemplary analysis are shown in FIG. 1A. The absorbed sera with high reactivity were incubated with PBMC from TKO pigs expressing Class I SLA and with PBMC from Class I SLA/αGal (GGTA, α1,3-galactosyl-transferase) knockout pigs. Binding to the different targets varied more than 25% in some samples, whereas others demonstrated minimal changes in reactivity regardless of the presence or absence of Class I SLA molecules. Nine of the 10 high binders showed reduced interaction with SLA KO cells. Data from an exemplary analysis are shown in FIG. 1B.

Serum from highly sensitized patients interacts with a broad spectrum of HLA alleles making these patients difficult to transplant. Sera from more than 15 patients with known calculated panel reactive antibodies (cPRA) greater than 80 were depleted with wild-type (VVT) red blood cells (RBC). The sera were incubated with αGal-knockout/SLA Class 1-negative and triple knockout out (TKO, CMAH, αGal, β4GalNT2 knockout), SLA Class 1-positive cells. Flow cytometry determined mean fluorescence indices (MFI) of binding to these cells for IgG and IgM. Data from representative assays are shown in FIG. 1C (IgG) and FIG. 1D (IgM). Thirteen of 22 highly sensitized patients contained Class I-SLA specific IgG; four of 17 highly sensitized patients contained Class I-SLA specific IgM.

### Example 2. Expression of SLA Alleles in C1R Cells

The indicated SLA alleles (SLA-1*0702, SLA-1*1201, SLA-1*1301, SLA-2*0202, SLA-2*1001, SLA-3*0402 and SLA-3*0502), were cloned into the pREP4 Mammalian Expression Vector (Invitrogen, Carlsbad Calif.). Human B-LCL (C1R) cells (unmodified), and SLA deficient pig renal endothelial cells were transfected with expression vectors comprising individual SLA-1 alleles. Stable transfectants were selected using antibiotic resistance selection; cell lines were maintained with hygromycin selection at 200 μg/mL. Transformed cells were incubated with monoclonal antibodies to either SLA or β2M to assess expression. Binding was analyzed by flow cytometry. Representative traces are shown in FIG. 2.

### Example 3. Creation of Modified C1R Cell Line with Reduced Antigenicity

The B cell Fc receptor gene was also targeted. Human B-LCL (C1R) cells were modified to disrupt the CIITA, B cell Fc receptor, and IgG genes using either the Crispr-Cas9 system or gRNA. Expression of targeted genes was evaluated. The resulting cells show reduced antigenicity to human sera.

CIR (HMy2.CIR) B lymphoblast cells were obtained from ATCC (C1R, HMy2.C1R, ATCC CRL-1993). (15) Autologous Class II, low levels of Class I, B-cell IgG receptor, and Fc-Receptor expression mitigated use as a human antibody target cell. The Crispr-Cas9 system was used to modify Human B-LCL (C1R) cells. Conserved segments of heavy chains of respective molecules were targeted, Table 1, using pSpCas9(BB)-2A-GFP (PX458) which was a gift from Feng Zhang (Addgene plasmid #48138). gRNA were designed to target IgG-1, IgG-2, IgG-3 and IgG-4. gRNA were designed to target HLA-DRα, HLA-DRβ, HLA-DQβ and HLA-DPβ. gRNA were designed to the Class I HLA heavy chain gene. The B cell Fc receptor gene was also targeted. Cells were stained with antiB2M antibody, Anti-Class II, anti-IgG and anti-FcyRIIb and cells were sorted for low expression. Lastly cells were incubated in animal serum free conditions and non-specific IgM antibody binding was blocked with Goat serum or inert serum incubation. B2m-02 sc-51510 (Santa Cruz), Jackson Immuno Goat-anti Mouse IgG F(ab′)2, HLA-DR/DP/DQ (WR18) FITC (Invitrogen), Anti-FcyRIIb/c clone 4F5, Goat antihuman IgG F(ab′)2, Donkey Anti-Human F(ab′)2 IgM. Results of phenotypic analysis of modified C1R cells are shown in FIGS. 14A-14C.

TABLE 1
CRISPR/Cas9 Guide RNA. The guide RNA's used to
target and disrupt protein expression in
the “naked” or modified C1R cells.

| | | SEQ |
|---|---|---|
| Gene family: | gRNA target sequence | ID NO: |
| Class I HLA | ″GACGCCGAGTTGCGGGTCA″ | 5 |
| | ″GACGCCGAGATGCGGGTCA″ | 6 |
| Class II HLA | ″GATGGTGCTGAGCTCCCCACTG″, | 7 |
| | ″GCCATAAGTGGAGTCCCTGTGCTAG″ | 8 |
| | ″GCGGATCCCCGGAGGCCTTCGG″ | 9 |
| | ″CCCCGGACAGTGGCTCTGACG″ | 10 |
| IgG | ″GGCCCATCGGTCTTCCCCC″ | 11 |
| | ″GTGACGGTGTCGTGGAACTC″ | 12 |
| | ″GCTCCAGGAGCACCTCTGG′ | 13 |
| | ″CCGAGAGCACAGCCGCCCT″ | 14 |
| Fc-receptor | ″ACTTCTTAGATTTTCCATTCTGG″ | 15 |

### Example 4. Expression of SLA alleles in Modified C1R Cells

Expression vectors containing SLA alleles (as described above herein) were transformed into modified reduced antigenicity C1R cells using methods similar to those for transforming unmodified C1R cells. Stable transfectants were selected using antibody resistance. Expression of the SLA allele is analyzed by any method known in the art. Human sera were incubated with modified C1R cells expressing SLA. Binding is evaluated by flow cytometry or any other method known in the art. See for example FIGS. 15A-15D.

### Example 5. Culture of Parent Pig Cell Line

A SV40 T antigen immortalized fibroblast cell line derived from a SLA Class I and galactose-α1,3-galactose deficient pig was cultured in minimum essential media (MEM-α) (Invitrogen, Carlsbad Calif.) supplemented with 10% fetal bovine serum and amphotericin B in collagen-I-coated plates at 37° C. and 5% CO₂. The genetically engineered αGal/3 classical Class I SLA gene knockout pig was previously obtained (Reyes et al 2014 J. Immunol. 193:5751-5757, herein incorporated by reference in its entirety). Cells were confirmed to be SLA Class II negative by incubation with anti-SLA-DR-FITC Ab or with anti-SLA-DQ-FITC (AbD Serotec, Raleigh N.C.) and analyzed using a BD Accuri C6 Flow Cytometer.

### Example 6. Creation of Pig Cell Line Expressing Class II SLA Molecules

Parent cells prepared as described above herein were grown to 90% confluency in a 10 cm culture plate and transfected with Lipofectamine 2000CD (Invitrogen) as specified by company protocol. A transgene encoding human CIITA was used to drive SLA Class II expression in the parent cell line. Downregulation of CIITA in human cells decreases expression of HLA Class 2 genes; human CIITA in porcine cells increases expression of SLA Class 2 genes. The donor plasmid was pCDNA3 myc CIITA (Addgene plasmid 14650). Three days post-transfection cells were screened on a BD Accuri C6 Flow Cytometer (BD Biosciences) using anti-SLA Class II DR-FITC Ab (AbD Serotec). Cells with high levels of Class II DR expression were sorted 1 cell per well into 96-well plates by the FACS Aria flow cytometer. The cells were placed into selection against Geneticin, G418 (Invitrogen). Expanded clonal cultures were then analyzed for presence or absence of SLA Class II DR using the previously mentioned anti-SLA Class II DR antibody. Clones with a high level of SLA Class II DR Ab binding were then evaluated for SLA Class II DQ (AbD Serotec). Two clones were selected: one that demonstrated a stable Class II positive (DR+/DQ+) phenotype and another with a Class II negative (DR−/DQ−) phenotype, both resistant to F418 selection. These cells contained the following Class II SLA genes: DRa (*02102/*w04re01), DRβ (1*1001/1*0403), DQα(*0204/*0101), DQβ (1*0601/1*0303). Representative results are shown in FIGS. 3A-3C. (See for example Reyes et al 2014, “Characterization of swine leukocyte antigen alleles in a crossbred pig to be used in xenotransplantation studies” Tissue Antigens, 84:484-488, herein incorporated by reference in its entirety).

### Example 7. Creation of Human Cells Expressing Class II SLA Molecules-Method 1 (Preferred)

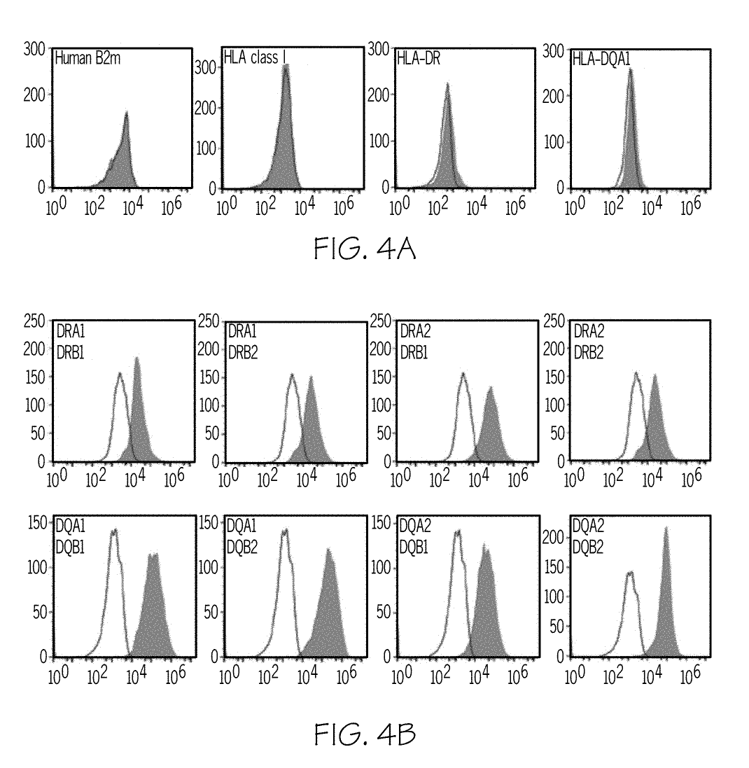

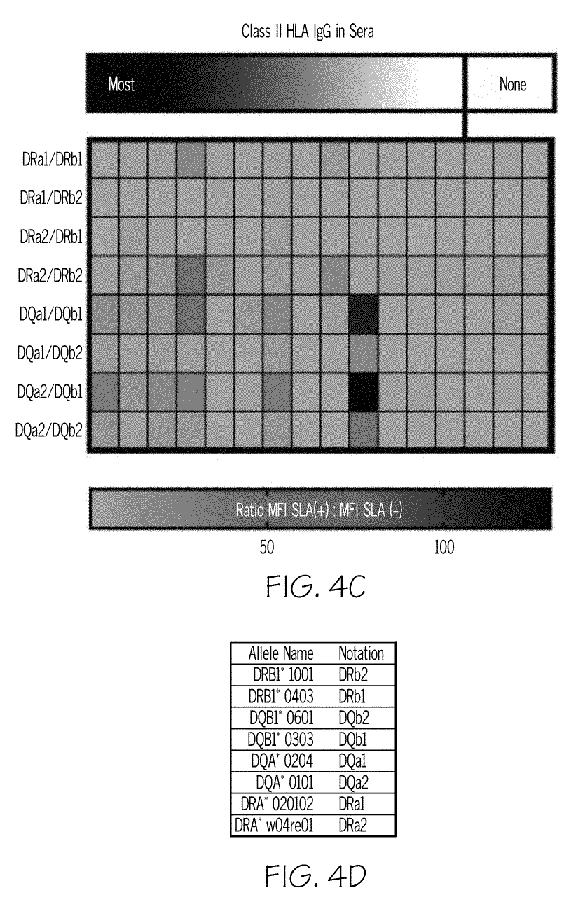

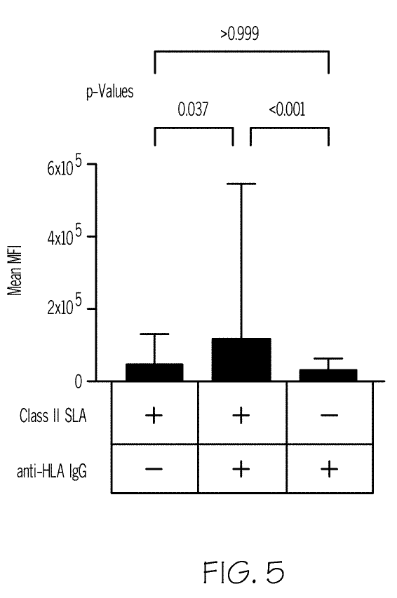

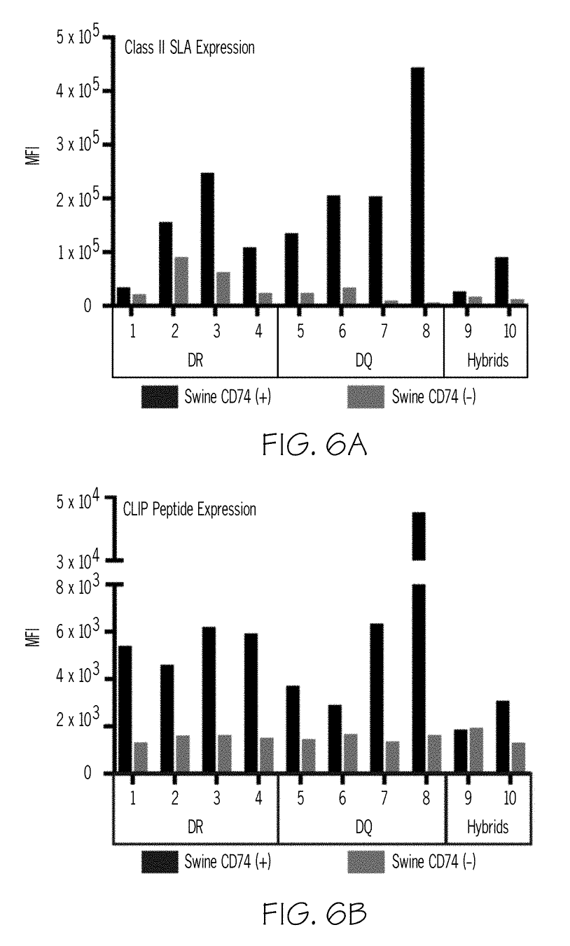

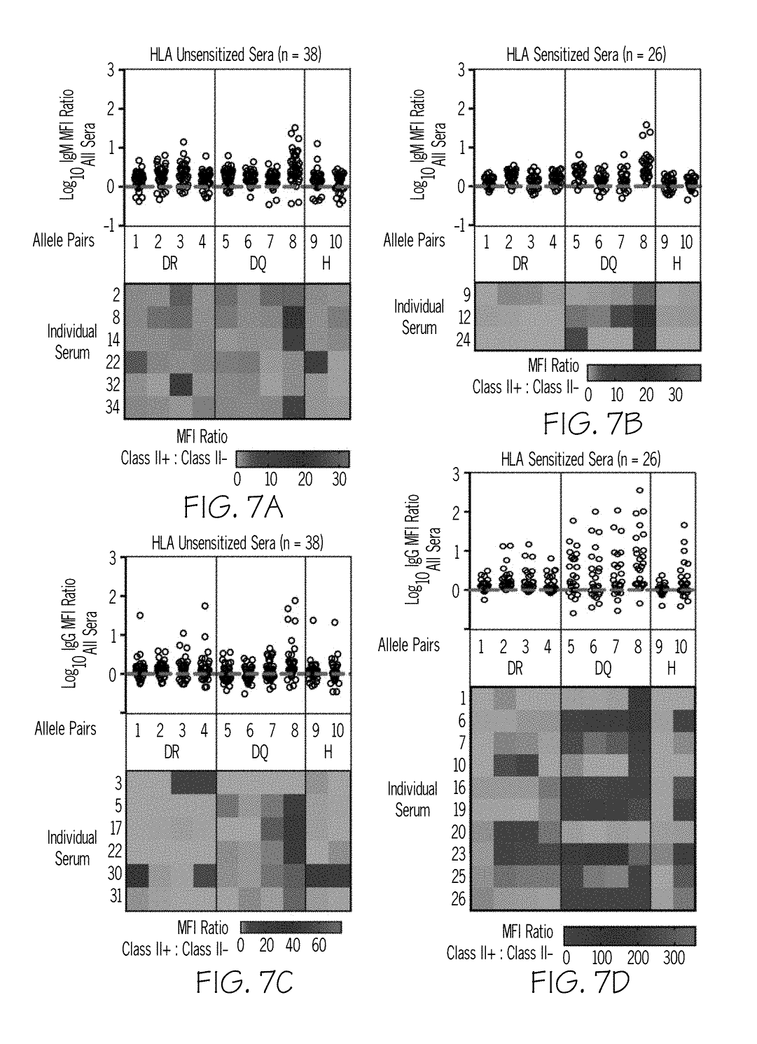

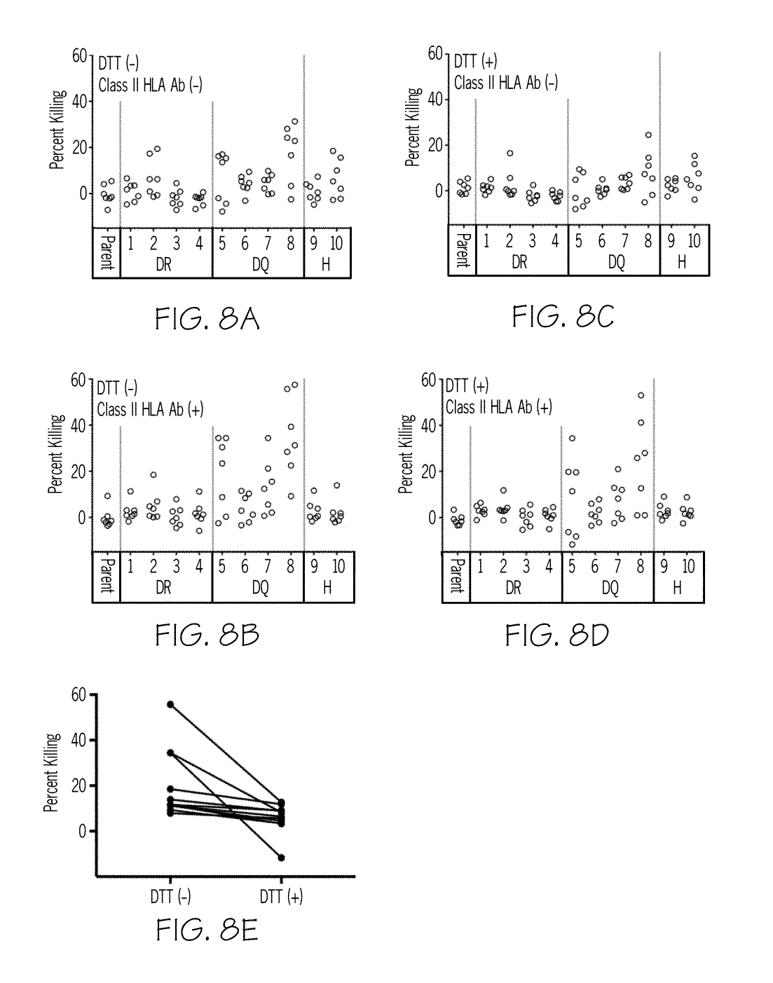

The expression vector, pBudCE4.1 (Thermo Fisher Scientific) was engineered to simultaneously express cDNA encoding SLA-DRA1-DRB1, -DRA1-DRB2, -DRA2-DRB1, -DRA2-DRB2, DQA1-DQB1, DQA1-DQB2, -DQA2-DQB1, or -DQA2-DQB2. These pairs of alpha and beta chains were from the 2 SLA haplotypes present in the fibroblasts used to obtain the results summarized in FIG. 5. The alpha chains of the alleles were inserted into the cytomegalovirus promoter site and the beta chains were inserted into the EF-1a site of pBudCE4.1 using restriction enzyme digestion and ligation. These plasmids were introduced into HEK 293T cells that had been made deficient in Class I HLA expression by using gRNA targeted to (forward: 5′-CTACTCTCTCTTTCTGGC-3′) set forth in SEQ ID NO:1 and (reverse: 5′-GGCCAGAAAGAGAGAGTAG-3′) set forth in SEQ ID NO:2 and the Crispr/Cas9 system to disrupt the β2-microglobulin gene. B2-microglobulin expression is known in the art to be critical for cell surface expression of Class I HLA. Class I HLA-deficient HEK cells were isolated by staining with anti-HLA Class I monoclonal antibody (Clone W6/32, ThermoFisherScientific) and sorting on a BD FCS Aria II at the UAB Comprehensive Flow Cytometry Core. Histogram traces of the parent cells incubated with various antibodies are shown in FIG. 4A. Once a HLA Class I-negative population was obtained, the cells were transfected using a calcium phosphate protocol. Briefly, 1×10⁶ cells were plated into a 6 well dish and transfected by adding a cocktail of 214 μl water, 31 μl 2 M CaCl₂, 2.5 μg DNA and 250 μl of 2×HBS. The cells were grown in MEM-α +10% fetal bovine serum+amphotericin. Three days post-transfection the cells were placed in selection with the antibiotic Zeocin (Thermo Fisher Scientific). The cells were sorted again at the UAB Comprehensive Flow Cytometry Core on a BED FACSAria II for SLA Class II expression as described above herein. Results from one series of experiments are shown in FIG. 4B.

### Example 8. Analysis of Pig CD74 Impact on SLA Class II Expression

Modified HEK293 cells lacking β2-microglobulin were transformed with an expression vector comprising pairs of SLA class II α and β chain alleles. Cell lines were also transformed with an expression vector comprising pig CD74. The SLA class II α and β chain alleles pairings are set forth in Table 2. Cell surface expression was analyzed with binding of a monoclonal antibody specific for Class II SLA and binding of CerCLIP.1, a monoclonal antibody specific for invariant chain peptides associated with Class II SLA. Ten different SLA molecules were created by expressing combinations of Class II SLA-DR and SLA-DQ α and β chains.

TABLE II
Allele combination nomenclature

| SLA Allele Combination | Pairing |
|---|---|
| DRα*020102 + DRβ1*0403 | 1 |
| DRα*020102 + DRβ1*1001 | 2 |
| DRα*w04 + DRβ1*0403 | 3 |
| DRα*w04 + DRβ1*1001 | 4 |
| DQα1*0204 + DQβ1*0303 | 5 |
| DQα1*0204 + DQβ1*0601 | 6 |
| DQα1*0101 + DQβ1*0303 | 7 |
| DQα1*0101 + DQβ1*0601 | 8 |
| DRα*020102 + DQβ1*0303 | 9 |
| DRα*w04 + DQβ1*0303 | 10 |

The various pairings of the α- and β-chains of DR and DQ are indicated. Pairing numbers will be used to simplify the labeling of figures.

Data and analysis from one such series of experiments are presented in FIGS. 12A-12B and 13A-13B.

### Example 9. Sequencing

PCR amplification of the Class II alleles was performed using the primers and conditions as described by Reyes et al. Ten PCR products were ligated into pCR2.1-TOPO plasmid using the TOPO TA cloning kit and analyzed by Sanger sequencing. Gel electrophoresis results are depicted in FIG. 3C.

### Example 10. Analysis of Human Antiporcine IgG Binding

One hundred and four human sera samples were obtained from discarded and de-identified material from the University of Alabama-Birmingham Histocompatability Lab were selected based on the presence or absence of Class II HLA-specific antibodies. The sera samples were heat inactivated at 57° C. for 30 minutes, and then sera were absorbed for 30 minutes with an equal volume of packed VVT pig red blood cells (RBC's) to reduce background binding by removing human antipig glycan antibodies.

Twenty-five microliters of absorbed human sera were incubated for 30 minutes at 4° C. with 1×10⁵ cells in EX-CELL 610-HSF Serum-Free Medium (Sigma, St. Louis Mo.) with 0.1% sodium azide on either the Class II positive or negative cell lines. Cells were washed 3 times with EX-CELL+sodium azide and then stained with goat anti-human IgG AlexaFlour 647 (Jackson ImmunoResearch Laboratories Inc., West Grove Pa.) for 30 minutes at 4° C. Cells were washed three times using EX-CELL medium as above and flow cytometric analysis was completed on BD Accuri C6 flow cytometer. Samples were gated on FSC-A by SSC-A. Statistical analysis was performed as described below herein.

### Example 11. Statistical Analysis of Antibody Binding

Human antibody binding results were reported as means of median fluorescence intensity (MFI) after subtracting out fluorescence values obtained with secondary antibodies alone. Graph and data analyses were completed using Prism 7 for Macintosh. The resulting data did not approximate normal distributions even after logarithmic transformation. Therefore the Kruskal-Wallis test was used to compare group means with correction by the Dunn multiple comparison test. Data from one such analysis is presented in FIG. 5.

### Example 12. Culture of Parent Human Embryonic Kidney 293T Cell Line-Method 2

Human embryonic kidney (HEK) 293T cells (CRL-3216; American Type Culture Collection) were cultured in minimum essential medium (MEM)-α supplemented with 10% FBS on collagen I-treated plates. Cells were incubated at 37° C. and 5% CO₂. Cells were confirmed to be Class II HLA-negative by incubation with an anti-HLA-DR antibody (clone L243) and anti-HLA-DQA1 (clone DQA1) and analyzed using BD Accuri C6 flow cytometer (BD Biosciences, San Jose Calif.).

### Example 13. Creation of β2-Microglobulin Deficient HEK293T Cells Expressing Class II SLA Molecules

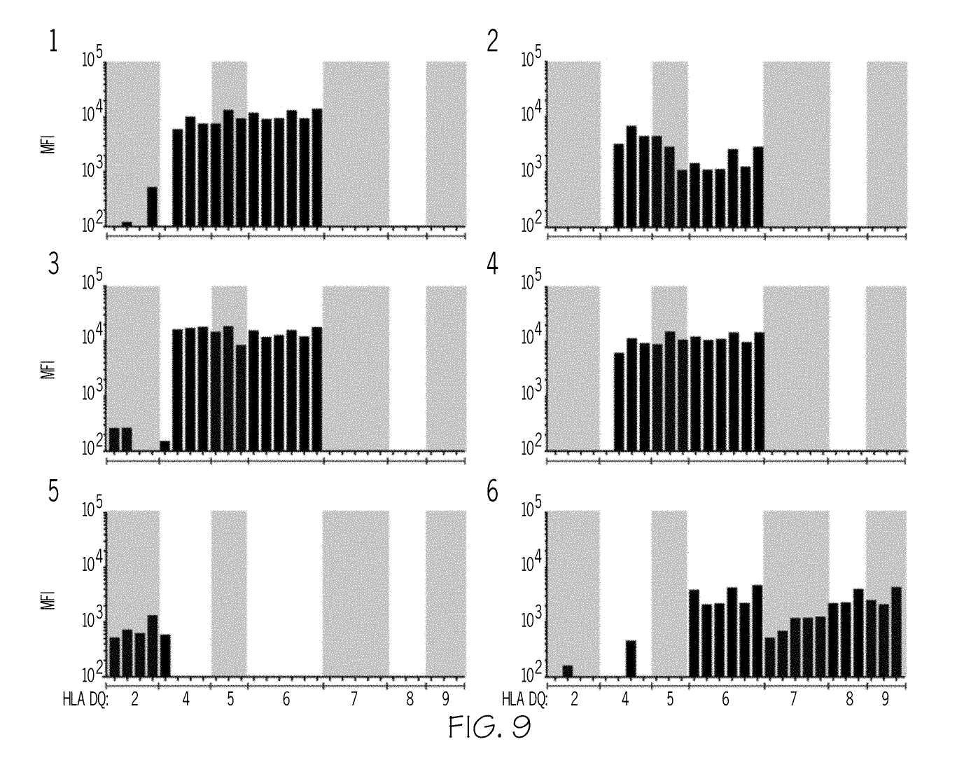

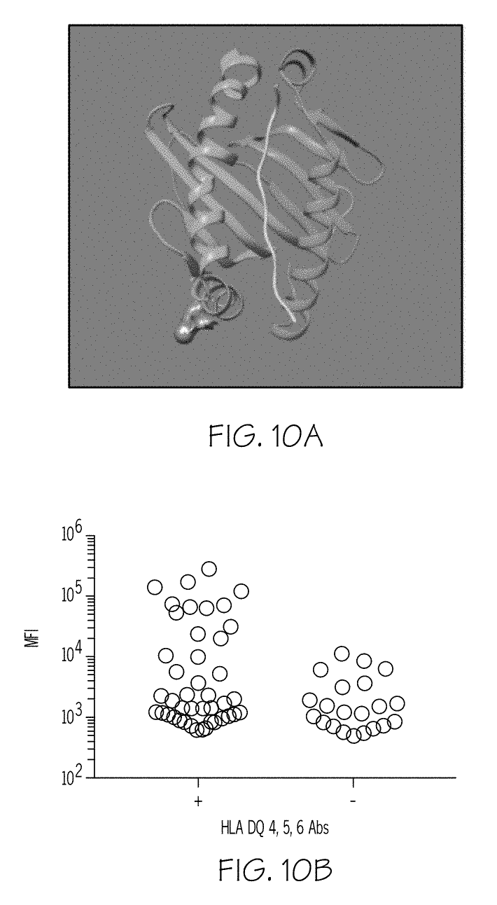

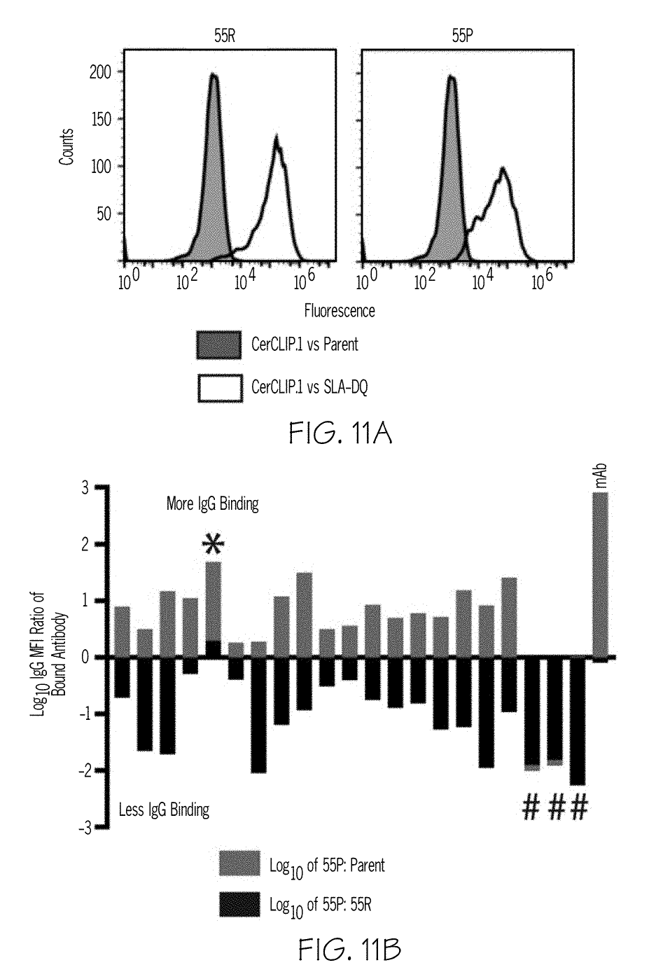

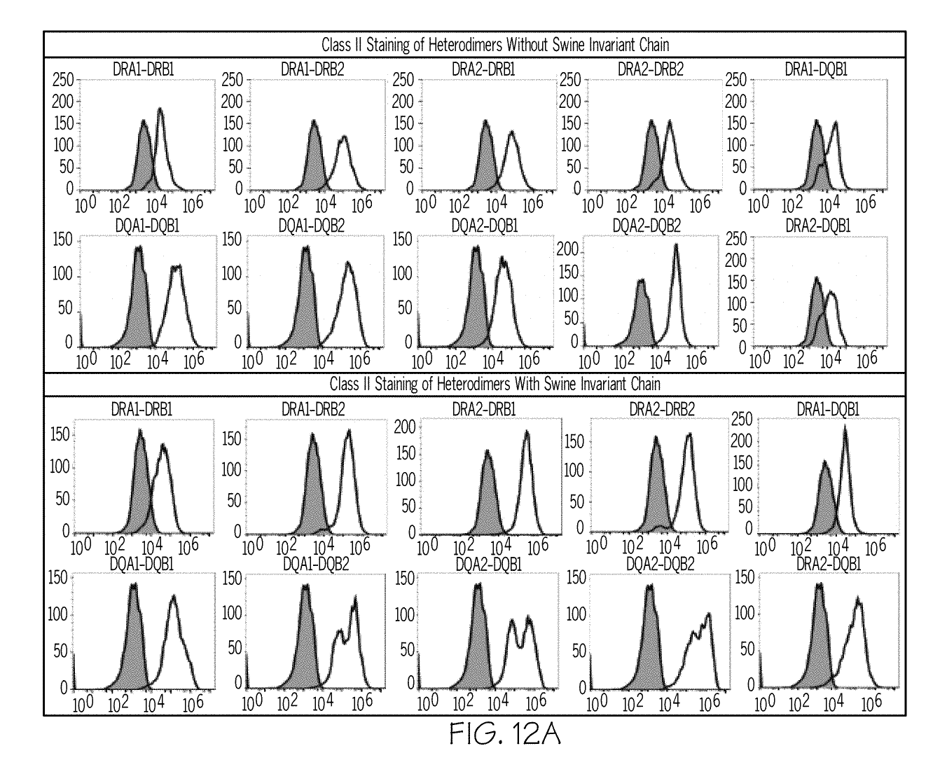

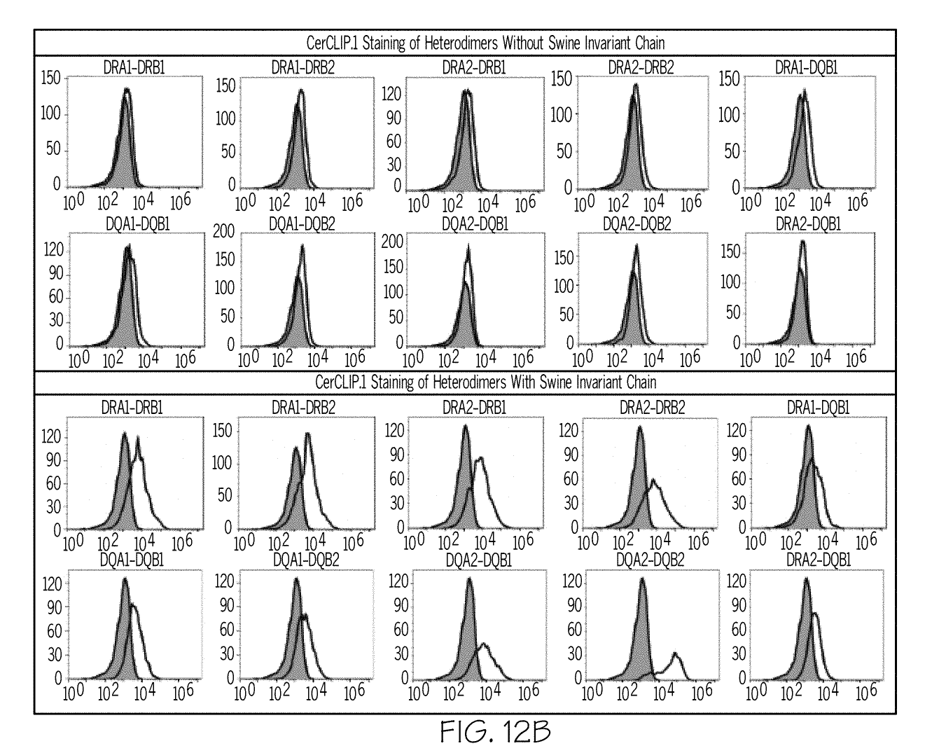

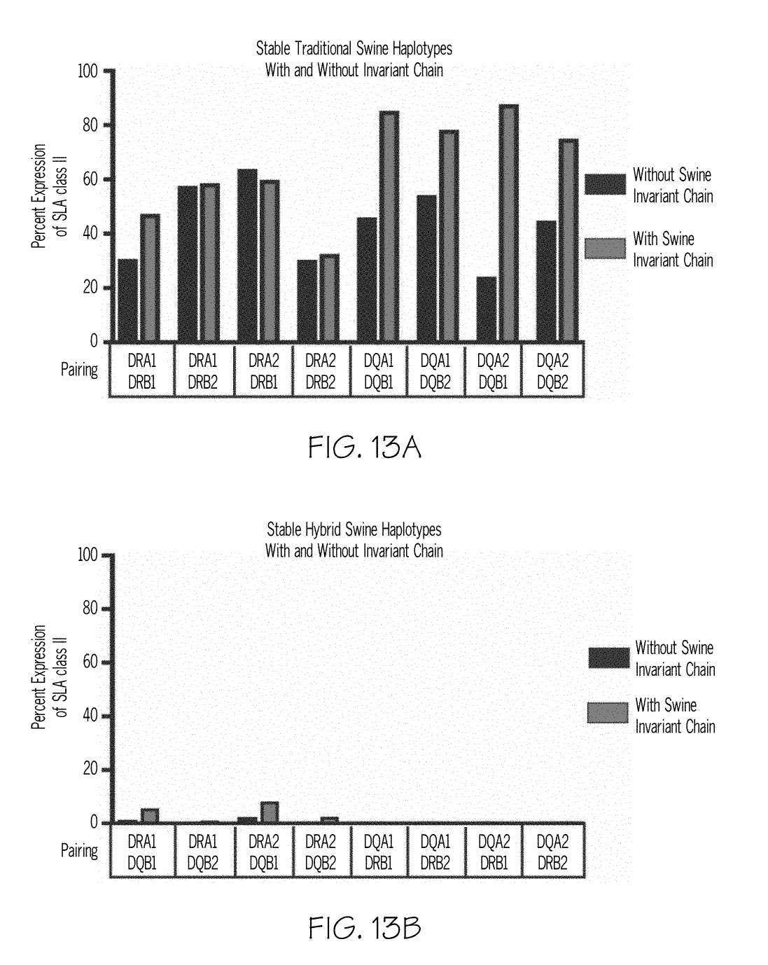

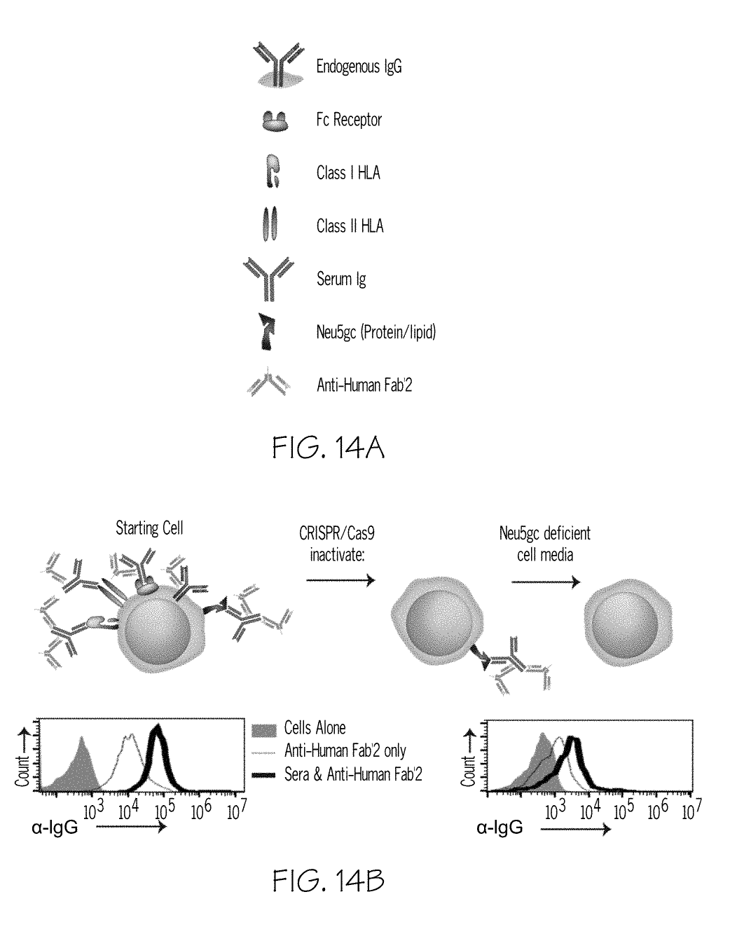

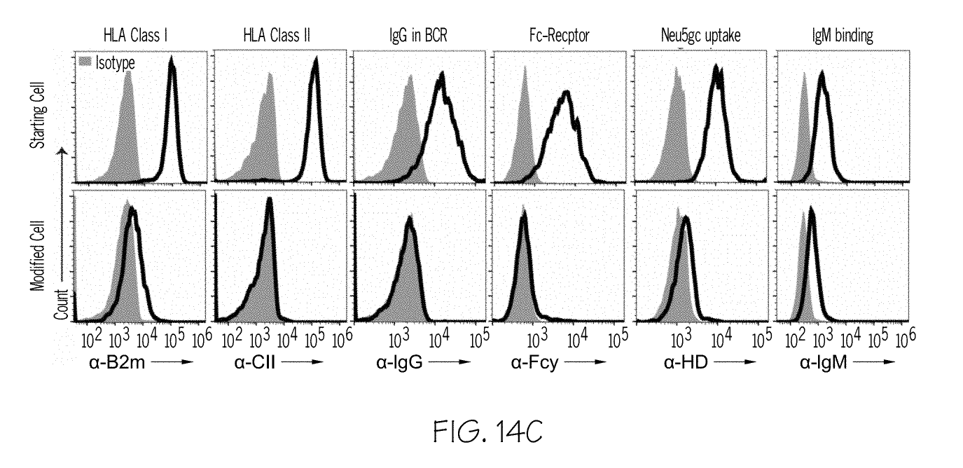

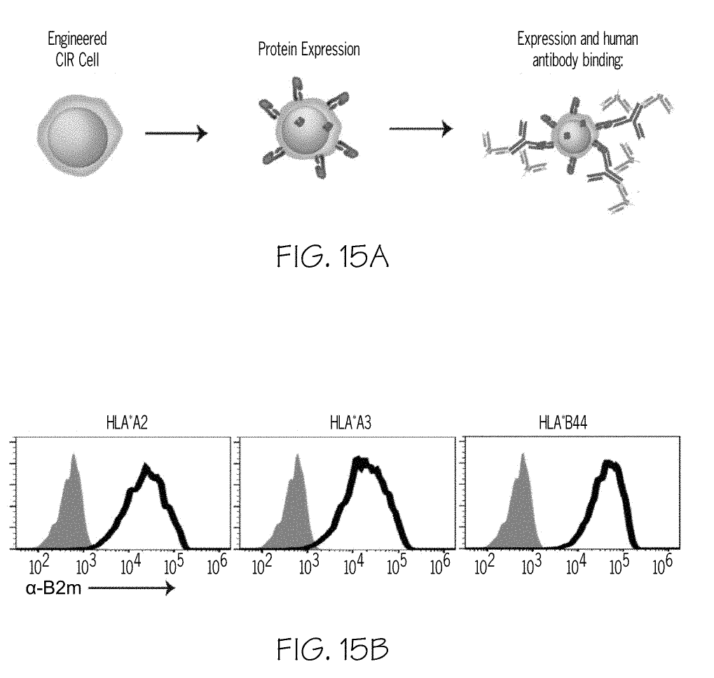

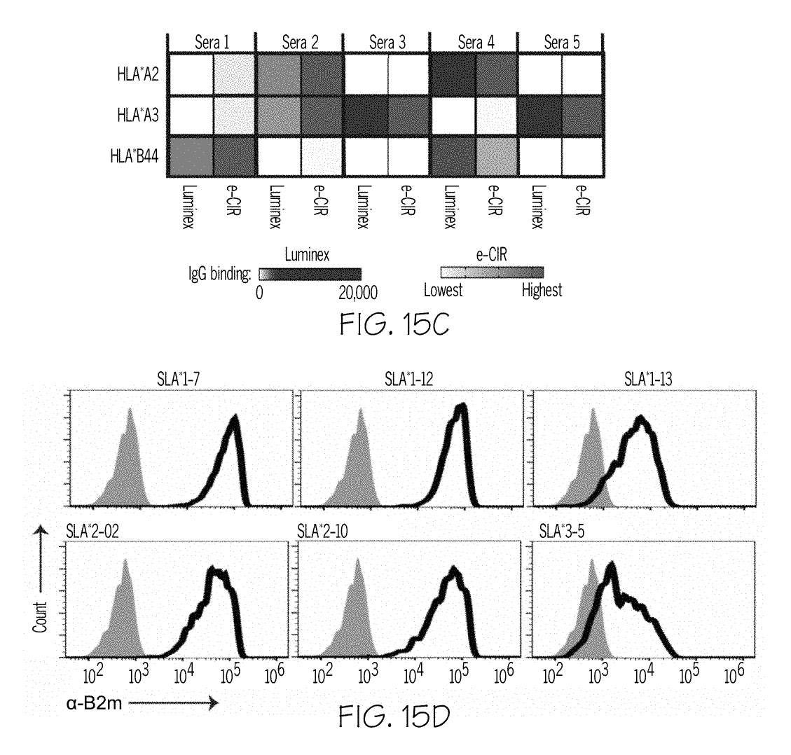

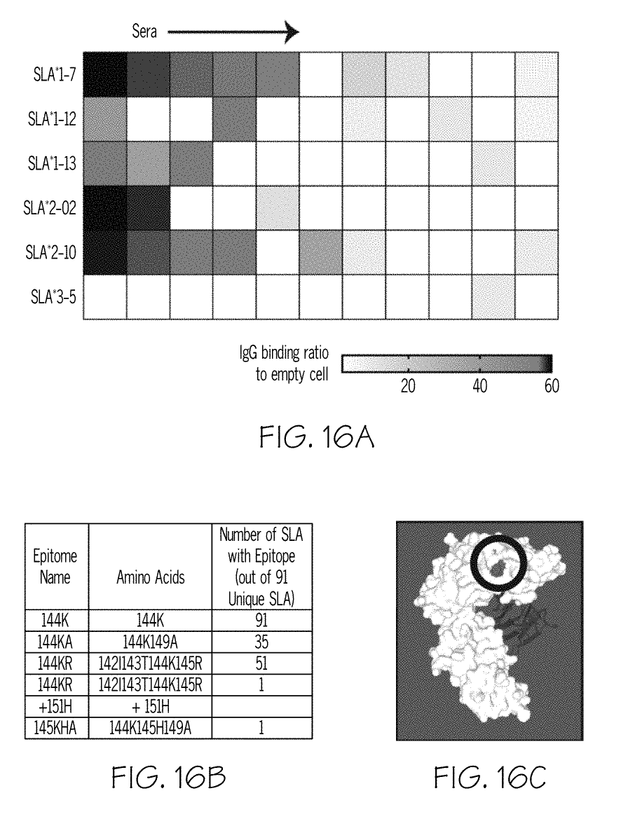

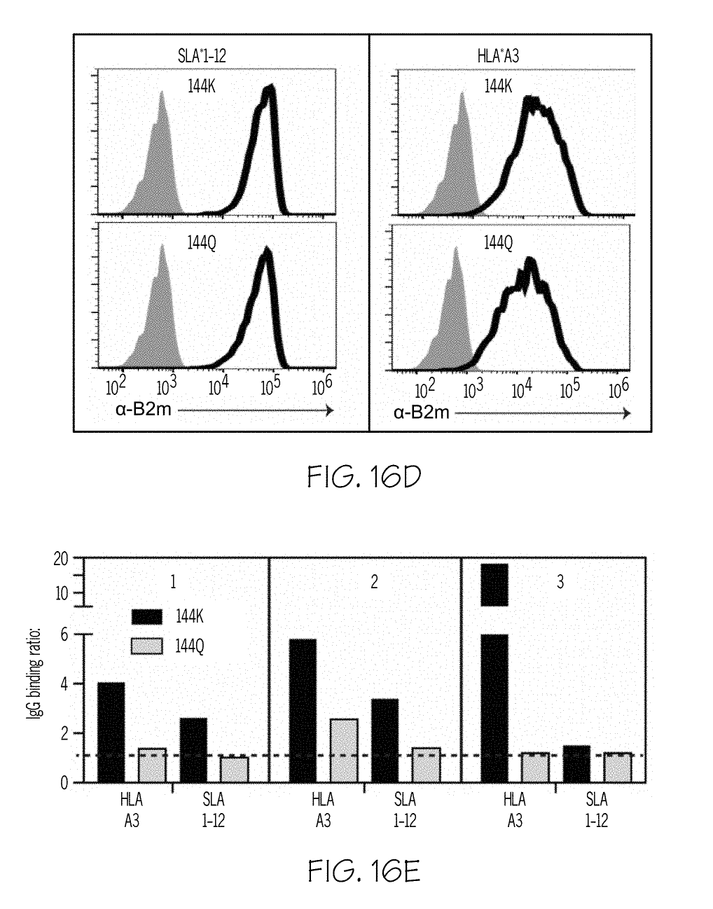

A bicistronic expression vector, pBudCE4.1, was engineered to express cDNA encoding pairs of 16 potential Class II SLA heterodimers. The plasmids were prepared as described above herein. A second plasmid pCDNA3.1+Hygro (Thermo Fisher Scientific) encoding the swine invariant chain was also created using restriction enzyme digestion and DNA ligation. These plasmids were introduced into modified HEK293T cells made deficient in class I HLA expression as described above herein. The Class I HLA-deficient cells were transfected using a calcium phosphate protocol: 1×10⁶ cells were plated into a six-well dish and transfected by adding a mixture of 214 μl water, 31 μl 2 M CaCl₂, 2.5 μg DNA and 250 μl of 2×HBS. The cells were placed into selection with either zeocin alone or zeocin and hygromycin for swine invariant chain transfectants, grown in MEM-α plus 10% FBS and sorted at the University of Alabama at Birmingham Comprehensive Flow Cytometry Core on a BD FACS Aria II for Class II SLA expression as described elsewhere herein. The sorted cells were analyzed by a monoclonal anti-CD74 (Cerclip.1) antibody (Novus Biologicals, Littleton Colo.). Results from one such experiment are presented in FIG. 6A-6B.

### Example 14. Complement-Dependent Cytotoxicity Assay on Class II SLA Single-Ag Cells

Sixty-four human sera samples, 26 sensitized toward Class II HLA and 38 with no sensitization, were chosen for study of haplotype-specific Ab binding on the panel of class II SLA single-Ag cells. Sera-treatment and methods of flow cytometric analysis were as described elsewhere herein. Results from one such experiment are presented in FIG. 7A-7D.

Fourteen of the 64 flow cytometry samples were screened by complement-dependent cytotoxicity. Seven had Class II HLA sensitization and seven lacked allosensitization. The complement-dependent cytotoxicity assay was modified from Diaz et at (2004), Cytometry A 62:54-60, herein incorporated by reference in its entirety. Cells (1×10⁵) were added to each well of Nunc 96-well polypropylene microwell plates (Thermo Fisher Scientific) in 25 μl HBSS and incubate at 4° C. for 30 minutes with 25 μl per well of human sera treated with or without DTT (2.5 mM in final) for 30 minutes at 37° C. The cells were washed three times with HBSS, then treated with 50 μl per well of an 11-fold dilution of Low-Tox-H rabbit complement (Cedarlane Laboratories, Burlington N.C.) for 90 minutes at 37° C. Cells were stained with fluorescein diacetate (0.5 μg/ml)/propidium iodide (2.5 mg/ml; Sigma-Aldrich) at 4° C. for 15 min. Data were collected using a BD Accuri C6 flow cytometer and software.

### Example 15. Ab Elution from Cells Expressing SLA-DQα1*0101 and DQβ1*0601

Six of the 64 flow cytometry samples were chosen for Ab binding and elution from a target cell. HEK293T β₂m-knockout cells (2×10⁶) expressing SLA-DQα1*0101, SLA-DQβ1*0601 and swine invariant chain (CD74) were incubated with 100 μl serum for 30 minutes at 4° C. Cells were washed twice with PBS to remove unbound antibodies. Citric acid/phosphate buffer (pH 3.3) was added for 2 minutes to elute bound Abs. Cells were pelleted by centrifugation and the supernatant was neutralized with TBS (pH 8) and concentrated to the original 100 μl of serum volume using Vivaspin 6 centrifugal (30, 000 Da molecular mass cutoff) concentrators. These samples were screened on a Class II HLA Luminex bead panel (One Lambda). Ab binding and elution from the HEK β₂-microglobulin (β₂m) knockout (KO) parent cell and rebinding to the Class II HLA Luminex bead panel were performed as a negative control.

### Example 16. Ab Binding of HLA-DQ4,5,6 Sensitized Sera on the SLA-DQ-Expressing Cell Line

Sixty-three Class II HLA-sensitized sera samples of discarded and deidentified material from the University of Alabama-Birmingham Histocompatibility Laboratory were provided based on the presence (42 samples) or absence (21 samples) of sensitization to HLA-DQ4,5, 6. Assays evaluating human Ab binding to cells expressing SLA-DQα1*0101, SLA-DQβ1*0601 and CD47 were performed. Results from one such experiment are presented in FIG. 10A-10B.

### Example 17. Creation of Mutated SLA-DQβ1*0601

The Arginine⁵⁵ residue of SLA-DQβ1*0601 was converted to Proline⁵⁵ in the pBUDCE4.1 SLADQα1*0101/SLA-DQβ1*0601 plasmid. A QuickChange XL site-directed mutagenesis kit (Agilent Technologies) was used with the following primers. forward, 5′-GTGAGTGACCCCGCTGGGGCCGCCGGACGCCGACTAC-3′ (SEQ ID NO:3); reverse, 5′-GTAGTCGGCGTCCGGCGGCCCCAGCGGGGTCAC-3′ (SEQ ID NO:4). HEK293T β₂m KO cells were transfected with the pBUDCE4.1 plasmid encoding DQα and the mutant DQβ and the pCDNA3.1+/Hygro plasmid, encoding the swine invariant chain with the calcium phosphate protocol described above herein. Transfected cells were placed into selection with zeocin and hygromycin, grown in MEM-α plus 10% FBS, and sorted on a BD FACSAria II for SLA Class II expression with the CerCLIP.1 mAb labeled with PE (clone CerCLIP.1; Thermo Fisher Scientific). A second monoclonal SLA-DQ Ab was also used to evaluate the SLA-DQ 55Pro mutant expression (clone TH81A5; Wash. State U.

### Example 18. Human Ab Binding to Cells Expressing SLA-DQα1*0101, 55P Mutant SLA-DQB1*0601 and CD74

Twenty-one sera samples from the cytotoxicity, elution and HLA-DQ4,5,6 sensitization studies suspected to have anti-SLA-Arg⁵⁵ Abs with sufficient volume for further analysis were tested for Ig binding to the parent β₂m KO HEK293T cells expressing SLA-DQα1*0101, 55PMutant SLA-DQβ1*0601 and CD74. Flow cytometry was performed as described above herein. Results from one such experiment are presented in FIG. 11B.

Flow cytometry files were analyzed in FlowJo v10 (Tree Star). Antibody binding results are reported as median fluorescent intensity (MFI) or ratio of MFI compared with MFI of sera on parent cells, as indicated in the figure description. Cytotoxicity killing and SLA Class II expression were determined by placing a fluorescent gate on the negative control. Graph and data analyses were completed using Prism7 for Macintosh (GraphPad Software). The resulting data did not approximate normal distributions even after logarithmic transformation. Therefore, the Wilcoxon and Mann-Whitney tests with correction by the Dunn multiple comparison tests were used to compare group means. SLA Class II molecule structure predictions were created in SWISS-MODEL and visualized in UCSF Chimera. See FIG. 10A.

### Example 19. Class II SLA Abs Drive Complement-Mediated Cytotoxicity

Parent cells were modified HEK293 cells lacking CD74 and Class II SLA. Modified cells were transfected with vectors expressing CD74 and pairs of Class II SLA alleles. Human sera were incubated with the panel of cells expressing various Class II SLA molecules and pig invariant chain 74. Rabbit complement was added and dead cells were counted as an indicator of Ab-initiated complement cytotoxicity. In certain instances DTT was added to inactivate IgM (see for example FIGS. 8A-8E).

### Example 20. Antibody Cross-Reactivity with Class II HLA and SLA Proteins

IgG specific for Class II HLA proteins were tested for cross-reactivity with Class II SLA. Modified HEK293 cells expressing CD74, SLA-DQα1*101, and DQβ1*0601 were used. These cells exhibit abundant cell surface Class II SLA protein. Six human sera were incubated with these cells. After incubation, cells were washed. Bound IgG antibodies were eluted and used to probe beads containing individual Class II HLA molecules. Data from one such experiment are summarized in FIG. 9.

### Example 21. Antibody Cross-Reactivity with R55P Class II SLA Proteins

The arginine at residue 55 of SLA-DQα1*01001/DQβ1*0601 was mutated to a proline (R55P) as described above herein. HEK293 cell lines expressing CD74, SLA-DQα1*0101 and mutant or unmodified SLA-DQβ1*0601 were created. Twenty-one human sera were incubated with cells expressing either the 55R or 55P variants of SLA-DQ. These sera were selected from prior experiments suggesting they reacted with SLA-DQα1*0101/SLA-DQβ1*0601. MFI ratios of fluorescent anti-human IgG were calculated to compare binding of 55P versus 55R molecules. The same analysis compared IgG binding to the 55P cells versus SLA-negative parent cells. Results from one such experiment are presented in FIG. 11A-11B.

### Example 22. Sensitized Patient Antibody Binding to Porcine PBMC, RBC and Modified-C1R Cells

Serum samples and data use were approved by the University of Alabama Institutional Review Board. Highly sensitized sera samples with varying reactivity to HLA-A, HLA-B and HLA-C groups were selected. Porcine PBMC's were isolated using Ficoll-Paque Plus (GE-Healthcare, Pittsburgh, Pa.) centrifugal separation and frozen in 90% autologous serum or stained immediately. RBC were collected and washed to remove buffy coat and platelets. Serum at 25% concentration was mixed with 2×10⁵ cells suspended in animal serum-free media with 0.1% sodium azide (EX-CELL 610-HSF, Sigma) for 30 minutes at 4° C. and washed three times. Bound human antibody was stained with goat anti-human Fab′2 IgG AlexaFlour 647 and donkey anti-human IgM Alexa Fluor 488 or 647 (Jackson Immunoresearch Laboratories Inc.) for 30 minutes at 4° C., washed twice and analyzed on an Attune NxT Flow cytometer or BD Accuri C6 flow cytometer. Similarly porcine RBC's were analyzed for porcine reactivity not attributed to SLA, which is absent or negligible on RBC cell surface. C1R cells were stained using 1×10⁵ cells and first incubation with serum was completed with addition of Goat serum blocking to prevent non-specific human IgM binding.

### Example 23. Porcine Reactive Antibody Elution and Single Antigen Screening

Patient sera were screened for PBMC binding. Samples with sufficient volume were incubated with PBMC from GGTA1/CMAH/B4GalNT2 triple knockout pigs at 1×10⁶ cells per 1 μL serum for 30 minutes at 4° C., washed twice, eluted with pH 3.3 citric acid/phosphate buffer, and neutralized with Tris-buffered saline as previously described. (7, 14) Reactivity to HLA was tested using LABScreen single-antigen HLA Class I beads (One Lambda Inc., Canoga Park, Calif.) and tested with Luminex analysis system (Luminex, Austin, Tex.). Data from one such series of experiments are included in FIGS. 15A-15D.

### Example 24. Site Directed Mutagenesis of SLA1-12

The QuickChange II Site Directed Mutagenesis Kit and an oligonucleotide with the following sequence were used to make a 144K to Q mutation in SLA1-12. The oligonucleotide sequence is set forth in SEQ ID NO:16; the sequence is 5′-CGGCUCAGAUCACCCAGCGCAAGUGGGAGG-3′. Position 144 is an important amino acid for multiple human cross-reactive epitope groups (CREGs) Glutamine/Q is the most common non-antigenic amino acid expressed at this location in other alleles. Position 144 may be a target for mutation in pigs. Relevant information is presented in FIGS. 16A-16E.

### Example 22. Generation of SLA-DQ/CMAH/GAL/β4GalNT2 Knockout Pig

Methods of producing multiple knockout pigs are known in the art and described in U.S. Pat. No. 9,888,674; U.S. patent application Ser. Nos. 15/520,633 and 15/739,469. Any method of making a knockout or transgenic pig may be used to make the quadruple knockout of the application. Commercially available Perv-C negative, blood group O pigs are purchased from Prestige Farms of Mississippi. Pairs of gRNA's with different target sequences are designed to target the SLA-DQ, CMAH, Gal and β4GalNT2 genes. Target sequences that are 20 to 300 bases apart, or preferably 20 to 50 bases apart are selected. These gRNA's may be obtained from a service such as Synthego. Each gRNA is mixed with recombinant Cas9 separately. The eight Cas9/gRNA complexes are then electroporated into ear fibroblasts isolated and cultured from Perv-C negative, blood group O pigs. Transfected cells are counter-selected with IB4 lectin. IB4 interacts with the Gal antigen. Cells that bind IB4 produce Gal; cells that lack the Gal product do not bind IB4. Cells that survive IB4 counter-selection are cloned. Quadruple knockouts are confirmed via DNA sequencing of the target regions. Validated cloned knockout cells are transferred into enucleated oocytes. Oocytes are transferred into a recipient pig and allowed to develop into either fetuses or live births.

That which is claimed:

1. An isolated modified C1R cell comprising a disrupted CIITA gene, a disrupted B cell Fc receptor gene and at least one disrupted IgG gene, wherein expression of MHCII, B cell Fc receptor and an IgG are reduced.

2. An isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and an IgG are reduced, and wherein said cell exhibits reduced antigenicity to human serum.

3. The isolated modified C1R cell of claim 2 further comprising at least two disrupted IgG genes or at least two disrupted Class II HLA α or β chain genes.

4. The isolated modified C1R cell of claim 2 wherein said disrupted IgG gene is selected from the group comprising IgG-1, IgG-2, IgG-3 and IgG-4 and wherein said disrupted Class II HLA α or β chain gene is selected from the group comprising HLA-DRα, HLA-DRβ, HLA-DQβ and HLA-DP-β.

5. A cell line comprising an isolated modified C1R cell of claim 2.

6. The isolated modified C1R cell of claim 2, wherein said isolated cell further comprises an expression vector encoding an antigen of interest, wherein expression of said antigen of interest is increased as compared to expression of said antigen of interest by an isolated modified C1R cell absent said expression vector.

7. The isolated modified C1R cell of claim 2, wherein said antigen of interest is selected from the group comprising Class I SLAs, Class II SLAs and HLAs.

8. The isolated modified C1R cell of claim 7, wherein said Class I SLA is selected from the group comprising SLA-1, SLA-2, SLA-3, SLA-5, SLA-6, SLA-7, SLA-8, SLA-12, SLA-1*0702, SLA-1*1201, SLA-1*1301, SLA-2*0202 and SLA-2* 1001, and said Class II SLA is selected from the group comprising SLA-DQ and SLA-DR.

9. A panel comprising a first and second isolated modified C1R cell of claim 2, wherein said first isolated modified C1R cell comprises a first expression vector encoding a first antigen of interest, and wherein said second isolated modified C1R cell further comprises a second expression vector encoding a second antigen of interest wherein expression of said first antigen of interest in said first cell and expression of said second antigen of interest in said second cell are increased as compared to expression of said antigens of interest by a modified cell of claim 2 absent an expression vector.

10. The panel of 11, further comprising a third isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and an IgG are reduced, and wherein said cell exhibits reduced antigenicity to human serum, wherein said third modified C1R cell further comprises a third expression vector encoding a third antigen of interest, wherein expression of said third antigen of interest is increased as compared to expression of said antigen of interest by an isolated modified C1R cell absent said expression vector.

11. The panel of 12 comprising a plurality of isolated modified C1R cells comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and an IgG are reduced, and wherein said cell exhibits reduced antigenicity to human serum, wherein said plurality of isolated modified cells each further comprises an expression vector encoding a different antigen of interest.

12. A method for detecting antibodies that bind an antigen of interest comprising the steps of:
(a) contacting a sample with a composition comprising an isolated modified cell of claim 2, wherein said isolated modified cell further comprises an expression vector encoding an antigen of interest wherein expression of said antigen of interest is increased as compared to expression of said antigen of interest by an isolated C1R cell absent said expression vector, and
(b) detecting binding of an antibody to said cell, wherein binding of an antibody to said cell is indicative of a sample comprising antibodies specific for said antigen of interest.

13. The method of claim 12, wherein said sample is a serum sample.

14. The method of claim 12, wherein said sample is obtained from a human subject that is a transplant candidate, a transplant recipient or transfusion recipient.

15. An isolated modified HEK293 T cell comprising a disruption of the β2-microglobulin gene wherein said cell exhibits reduced Class I HLA expression, further comprising a first expression vector encoding a first antigen of interest, wherein expression of said first antigen of interest is increased as compared to expression of said first antigen of interest by an isolated modified HEK293 T cell comprising a disruption of the β2-microglobulin gene wherein said cell exhibits reduced Class I HLA expression absent said expression vector.

16. The isolated modified HEK293T cell of claim 15, wherein said first expression vector further encodes a second antigen of interest, wherein expression of said second antigen of interest is increased as compared to expression of said second antigen of interest by an isolated modified HEK293T cell with reduced Class I HLA expression absent said expression vector.

17. The isolated modified HEK293 T cell of claim 15, further comprising a second expression vector encoding CD74, wherein expression of CD74 is increased as compared to expression of CD74 by an isolated modified HEK293 T cell with reduced Class I HLA expression absent said second expression vector.

18. The isolated modified HEK293 T cell of claim 15, wherein said antigen of interest is selected from the group comprising Class I SLAs, Class II SLAs and HLAs.

19. The isolated modified HEK293T cell of claim 18, wherein said Class I SLA is selected from the group comprising SLA-1, SLA-2, SLA-3, SLA-5, SLA-6, SLA-7, SLA-8, SLA-12 and said Class II SLA is selected from the group comprising SLA-DQ and SLA-DR.

20. The isolated HEK293T cell of claim 16, wherein said first antigen of interest is a Class II SLA α-chain and wherein said second antigen of interest is a Class II SLA β-chain.

21. The isolated HEK293T cell of claim 20 wherein said first and second antigens of interest are a pair of Class II SLA α- and β-chain antigens selected from the pairs of Class II SLA α- and β-chain antigens comprising DRα*020102 and DRβ1*0403, DRα*020102 and DRβ1*1001, DRα*w04 and DRβ1*0403, DRα*w04 and DRβ1*1001, DQα1*0204 and DQβ1*0303, DQα1*0204 and DQβ1*0601, DQα1*0101 and DQβ1*0303, DQα1*0101 and DQβ1*0601, DRα*020102 and DQβ1*0303, DRα*w04 and DQβ1*0303, DRA1 and DRB1, DRA1 and DRB2, DRA2 and DRB1, DRA2 and DRB2, DQA1 and DQB1, DQA1 and DQB2, DQA2 and DQB1, DQA2 and DQB2, DRA1 and DQB1, DRA1 and DQB2, DRA2 and DQB1, DRA2 and DQB2, DQA1 and DRB1, DQA1 and DRB2, DQA2 and DRB1, and DQA2 and DRB2.

22. A panel comprising a first isolated modified HEK293 T cell comprising a disruption of the β2-microglobulin gene wherein said cell exhibits reduced Class I HLA expression, wherein said first isolated cell further comprises a first expression vector encoding a first antigen of interest, and a second isolated modified HEK293 T cell comprising a disruption of the β2-microglobulin gene wherein said cell exhibits reduced Class I HLA expression wherein said second isolated cell further comprises a second expression vector encoding an additional antigen of interest, wherein expression of said antigens of interest is increased as compared to expression of said antigens of interest by an isolated modified HEK293 T cell comprising a disruption of the β2-microglobulin gene wherein said cell exhibits reduced Class I HLA expression absent said expression vectors.

23. The panel of claim 22 further comprising a third isolated modified HEK293 T cell comprising a disruption of the β2-microglobulin gene wherein said cell exhibits reduced Class I HLA expression, wherein said third isolated cell further comprises a third expression vector encoding another antigen of interest, wherein expression of said antigen of interest is increased as compared to expression of said antigen of interest by an isolated modified HEK293 T cell comprising a disruption of the β2-microglobulin gene wherein said cell exhibits reduced Class I HLA expression absent said expression vector.

24. The panel of claim 23 further comprising a plurality of isolated modified HEK293 T cells comprising a disruption of the β2-microglobulin gene cells wherein said cell exhibits reduced Class I HLA expression, wherein said isolated cells further comprise an expression vector encoding a plurality of different antigens of interest.

25. The panel of claim 24 wherein said isolated modified HEK293T cell comprising a disruption of the β2-microglobulin gene wherein said cell exhibits reduced Class I HLA expression further comprises a second expression vector encoding CD74, wherein expression of CD74 is increased as compared to expression of CD74 by an isolated modified HEK293T cell comprising a disruption of the β2-microglobulin gene wherein said cell exhibits reduced class I HLA expression.

26. A panel comprising a first isolated modified HEK293 T cell comprising a disruption of the β2-microglobulin gene wherein said cell exhibits reduced Class I HLA expression, wherein said first cell comprises a first expression vector encoding a first and a second antigen of interest, wherein expression of said first and second antigens of interest is increased as compared to expression of said second antigens by an isolated modified HEK293 T cell comprising a disruption of the β2-microglobulin gene wherein said cell exhibits reduced class I HLA expression, and a second isolated modified HEK293 T cell comprising a disruption of the β2-microglobulin gene wherein said cell exhibits reduced class I HLA expression, wherein said second isolated cell further comprises a expression vector encoding at least a third antigen of interest, wherein expression of said third antigen of interest is increased as compared to expression of said third antigen of interest by an isolated modified HEK293 T cell comprising a disruption of the β2-microglobulin gene wherein said cell exhibits reduced Class I HLA expression absent said expression vector.

27. The panel of claim 26, further comprising a third isolated modified HEK293 T cell comprising a disruption of the β2-microglobulin gene wherein said cell exhibits reduced class I HLA expression, wherein said third cell further comprises an expression vector encoding at least one additional antigen of interest, wherein expression of said additional antigen of interest is increased as compared to expression of said antigen of interest by an isolated modified HEK293 T cell comprising a disruption of the β2-microglobulin gene wherein said cell exhibits reduced Class I HLA expression absent said expression vector.

28. The panel of claim 27 further comprising a plurality of isolated HEK293Tcells comprising a disruption of the β2-microglobulin gene wherein said cell exhibits reduced Class I HLA expression, wherein said plurality of isolated cells each further comprises an expression vector encoding a different antigen of interest.

29. The panel of claim 26 wherein said first and second antigens of interest are a pair of class II SLA α- and β-chain antigens selected from the pairs of class II SLA α- and β-chain antigens comprising DRα*020102 and DRβ1*0403, DRα*020102 and DRβ1*1001, DRα*w04 and DRβ1*0403, DRα*w04 and DR β1*1001, DQα1*0204 and DQβ1*0303, DQα1*0204 and DQβ1*0601, DQα1*0101 and DQβ1*0303, DQα1*0101 and DQβ1*0601, DRα*020102 and DQβ1*0303, DRα*w04 and DQβ1*0303, DRA1 and DRB1, DRA1 and DRB2, DRA2 and DRB1, DRA2 and DRB2, DQA1 and DQB1, DQA1 and DQB2, DQA2 and DQB1, DQA2 and DQB2, DRA1 and DQB1, DRA1 and DQB2, DRA2 and DQB1, DRA2 and DQB2, DQA1 and DRB1, DQA1 and DRB2, DQA2 and DRB1, and DQA2 and DRB2.

30. A panel comprising: (a) at least one isolated modified C1R cell comprising a disrupted Class I HLA heavy chain gene, a disrupted B cell Fc receptor gene, at least one disrupted Class II HLA α or 3 chain gene, and at least one disrupted IgG gene, wherein expression of Class I HLA, MHCII, B cell Fc receptor and an IgG are reduced, and wherein said cell exhibits reduced antigenicity to human serum, further comprising a first expression vector encoding a first antigen of interest, wherein expression of said first antigen of interest is increased as compared to expression of said first antigen of interest by an isolated modified C1R cell comprising a disrupted Class I HLA heavy chain gene, a disrupted B cell Fc receptor gene, at least one disrupted Class II HLA α or β chain gene, and at least one disrupted IgG gene, wherein expression of Class I HLA, MHCII, B cell Fc receptor and an IgG are reduced, and wherein said cell exhibits reduced antigenicity to human serum absent said expression vector and (b) at least one isolated modified HEK293 T cell comprising a disruption of the β2-microglobulin gene wherein said cell exhibits reduced Class I HLA expression, further comprising a second expression vector encoding a second antigen of interest and wherein expression of said second antigen of interest is increased as compared to expression of said second antigen of interest by an isolated modified HEK293T cell comprising a disruption of the β2-microglobulin gene wherein said cell exhibits reduced Class I HLA absent said second expression vector.

31. A method for detecting antibodies that bind an antigen of interest comprising the steps of: (a) contacting a sample with a composition comprising an isolated modified HEK293T cell comprising a disruption of the β-2 microglobulin gene wherein said cell exhibits reduced Class I HLA expression, further comprising an expression vector encoding an antigen of interest wherein expression of said antigen of interest is increased as compared to expression of said antigen of interest by an isolated modified HEK293T cell comprising a disruption of the β-2 microglobulin gene wherein said cell exhibits reduced Class I HLA expression absent said expression vector, and (b) detecting binding of an antibody to the cell, wherein binding of an antibody to the cell is indicative of a sample comprising antibodies specific for the antigen of interest.

32. A transgenic pig comprising a disrupted α(1,3)-galactosyltransferase, CMAH, β4-galactosyltransferase (β4GalNT2) and SLA gene in the nuclear genome of at least one cell of said pig, wherein expression of α(1,3)-galactosyltransferase, CMAH, β4GalNT2 and SLA is decreased as compared to a wild-type pig.

33. The transgenic pig of claim 32, wherein said disrupted SLA gene is a disruption of the SLA-DQ gene selected from the group of SLA-DQ gene disruptions comprising truncations, insertions, deletions, substitutions at position 55, and a proline substitution at position 55.


SEQUENCE LISTING (16 sequences)

SEQ ID NO: 1 - DNA, 18 bp, Homo sapiens
ctactctctctttctggc

SEQ ID NO: 2 - DNA, 19 bp, Homo sapiens
ggccagaaagagagagtag

SEQ ID NO: 3 - DNA, 37 bp, Homo sapiens
gtgagtgaccccgctggggccgccggacgccgactac

SEQ ID NO: 4 - DNA, 33 bp, Homo sapiens
gtagtcggcgtccggcggccccagcggggtcac

SEQ ID NO: 5 - DNA, 19 bp, Homo sapiens
gacgccgagttgcgggtca

SEQ ID NO: 6 - DNA, 19 bp, Homo sapiens
gacgccgagatgcgggtca

SEQ ID NO: 7 - DNA, 22 bp, Homo sapiens
gatggtgctgagctccccactg

SEQ ID NO: 8 - DNA, 25 bp, Homo sapiens
gccataagtggagtccctgtgctag

SEQ ID NO: 9 - DNA, 22 bp, Homo sapiens
gcggatccccggaggccttcgg

SEQ ID NO: 10 - DNA, 21 bp, Homo sapiens
ccccggacagtggctctgacg

SEQ ID NO: 11 - DNA, 19 bp, Homo sapiens
ggcccatcggtcttccccc

SEQ ID NO: 12 - DNA, 20 bp, Homo sapiens
gtgacggtgtcgtggaactc

SEQ ID NO: 13 - DNA, 19 bp, Homo sapiens
gctccaggagcacctctgg

SEQ ID NO: 14 - DNA, 19 bp, Homo sapiens
ccgagagcacagccgccct

SEQ ID NO: 15 - DNA, 23 bp, Homo sapiens
acttcttagattttccattctgg

SEQ ID NO: 16 - RNA, 30 nt, Artificial Sequence
  site directed mutagenesis oligo
cggcucagaucacccagcgcaagugggagg